(19) United States
(12) Patent Application Publication
(10) Pub. No.: US 2020/0163896 A1
(43) Pub. Date: May 28, 2020

(54) FLOATABLE PHARMACEUTICAL MICROCAPSULE COMPOSITION

(71) Applicant: Nanyang Technological University, Singapore (SG)

(72) Inventors: Say Chye Joachim LOO, Singapore (SG); Jongsuep BAEK, Singapore (SG)

(21) Appl. No.: 16/635,310

(22) PCT Filed: Aug. 2, 2018
(86) PCT No.: PCT/SG2018/050394

§ 371 Date: Jan. 30, 2020

(30) Foreign Application Priority Data
Aug. 2, 2017 (SG) 10201706305X

(51) Int. Cl. A61K 9/50; A61K 47/34; A61K 47/42; A61K 47/44; A61K 31/198; A61K 31/155; A61K 31/216; A61K 31/277; A61K 31/4409; A61K 31/496; A61K 31/133

(52) U.S. Cl. CPC A61K 9/5084; A61K 47/34; A61K 47/42; A61K 47/44; A61K 31/198; A61K 9/5089; A61K 31/216; A61K 31/277; A61K 31/4409; A61K 31/496; A61K 31/133; A61K 31/155

(57) ABSTRACT

Disclosed herein is a sustained release hollow core-shell microcapsule formulation for drug delivery comprising a hollow shell of one or more hydrophobic polymers, a drug containing hydrophilic or amphiphilic carrier matrix that is distributed over the inner surface of the hollow shell, a flotation agent, and an optional osmotic agent, wherein the microcapsule is capable of floating in a simulated digestive fluid for a period of from 24 to 96 hours. The microcapsules are prepared by a modified double emulsion (water/oil/water) solvent evaporation method. The microcapsule formulation may provide a sustained-release delivery system for treating chronic diseases such as Parkinson's disease, diabetes and tuberculosis, all of which require multiple drug combination therapies. The aim is to reduce dosing frequency and pill burden, thus improving patient medication compliance. In a specific embodiment, the hollow shell is formed from a mixture of Poly-L-lactide (PLLA) and poly(E-caprolactone) (PCL); and the amphiphilic carrier matrix is casein.

### FIELD OF INVENTION

The invention relates to gastric-floating microcapsules entrapping one or more, preferably multiple, drugs and a method to fabricate the same.

### BACKGROUND

The listing or discussion of an apparently prior-published document in this specification should not necessarily be taken as an acknowledgement that the document is part of the state of the art or is common general knowledge.

Parkinson's disease (PD) is a degenerative disorder of the central nervous system, leading to asymmetric onset of bradykinesia, resting tremor, rigidity and postural instability. It is more common in older people, with most cases occurring after the age of 50 years. The early signs and symptoms may be mild and may go unnoticed initially, but symptoms of this motor-degenerative disease stem from the death of dopamine-generating cells in the substantia nigra, a region of the mid-brain.

Whilst the cause of Parkinson's disease remains uncertain, the mainstay of management is a pharmacological regimen. Although Parkinson's disease cannot be cured, management through the pharmacological approach, with medications taken over a prolonged period, do aid in controlling symptoms and thus improve daily function of these patients. In addition, studies have shown that medications can prevent further deterioration of the patient and provide neuroprotective effects. To achieve desirable management of Parkinson's disease, the patient is therefore required to commit religiously to the prescribed medication regimen. However, many patients find it difficult to be fully compliant because a large number of these are elderly, and are probably already taking multiple pills for other ailments. These elderly patients are also more likely to forget or “miss” their daily medications—the leading cause of patient-based medication noncompliance. There is therefore a need for improved dosage forms that reduces the need for frequent medication, so as to improve patient medication compliance.

Although levodopa (LD) is the most effective drug for treating PD, chronic administration of LD leads to a pharmacological problem, levodopa-induced dyskinesia (LID). It is widely accepted that LID is due, at least in part, to the short half-life of LD. Dyskinesia most commonly occurs at the time of peak LD plasma concentrations during intermittent or pulsatile LD stimulation and is thus referred to as peak-dose dyskinesia. To mitigate LID, a continuous and non-fluctuating provision of LD to the brain is therefore essential. Current oral formulations in the market are not able to provide this. Most patients are known to take up to five tablets a day, resulting in a sinusoidal rise and decline of LD, which is the cause of LID. Therefore, an improved dosage form that provides a controlled release of PD drugs would help to mitigate LID. The improved dosage form should ideally reduce the dosing of LD drugs to just once a day, or possibly even less frequently.

Examples of commercial combination products for the management of Parkinson's disease, include Sinemet™, Stalevo™, and Rytary™. Sinemet™ and Stalev™ are not sustained release formulations and so patients have to take these up to five times a day. All of these tablets contain the active ingredient LD. LD is converted into dopamine in the brain, replacing the lost dopamine. This reduces some of the symptoms associated with the disease. Other drugs are usually used synergistically with LD—and one or more of these is contained in the above-mentioned products. For instance, carbidopa (CD) is an inhibitor of aromatic amino acid decarboxylation. Entacapone (ENT), a catechol-O-methyltransferase inhibitor, is a nitro-catechol-structured compound. Both CD and ENT work synergistically to increase bioavailability of LD in the brain for conversion to dopamine. Although Rytary™ has sustained release capabilities, it contains only two of the three PD drugs, i.e., LD/CD.

Diabetic patients are also often treated with multiple drugs. Metformin (MET) is a first line therapeutic agent for Type II diabetes. MET increases insulin sensitivity and glucose tolerance by lowering both basal and postprandial glucose levels. In order to reduce incidences of cardiovascular events (i.e. myocardial infarction) that are associated with Type II diabetes, other drugs are also co-administered. For example, Fenofibrate (FEN) shows synergistic effects with MET as it enhances therapeutic effects and provides cardioprotection. As such, diabetes is another disease which can be better treated with an improved dosage form that provides controlled release of multiple drugs, so as to improve patient compliance and treatment outcomes.

Tuberculosis (TB) can be treated using a combination of different drugs over a course of up to six months. Although current TB treatment regimens can cure most patients who have TB, it is often suggested that TB treatment fails because patients do not take their TB drugs correctly, for example, when patients do not comply to the TB treatment for the full therapy duration. A patient who does not take his/her TB drug treatment properly can also lead to the development of drug resistant TB. As such, the use of an improved dosage form/drug delivery system that provides controlled or sustained release of multiple drugs may overcome some of these issues in the treatment of TB.

It is shown above that there are diseases which are treated by pharmacological regimes which require patients to take multiple drugs daily, or require patients to take drugs at a high daily frequency. There is therefore a need for an improved drug delivery system that overcomes some or all of the issues mentioned.

### SUMMARY OF INVENTION

The inventors have surprisingly found that a sustained release formulation comprising one or more active ingredients can help to overcome the issues of patient compliance and help to reduce issues associated with the concentration of the active ingredient(s) exceeding or being less than the therapeutic concentration window. Thus, in a first aspect of the invention, there is provided a sustained release hollow core-shell microcapsule formulation for drug delivery, comprising:
a hollow shell having an outer surface and an inner surface that is formed from one or more hydrophobic polymers;
a hydrophilic or amphiphilic carrier matrix distributed over the inner surface of the hollow shell;
a first drug distributed within the hydrophilic or amphiphilic carrier matrix;
optionally, an osmotic agent; and
a flotation agent, wherein
the microcapsule is capable of floating in a simulated digestive fluid for a period of from 24 to 96 hours.

In embodiments of the first aspect of the invention:

(A) the hydrophobic polymer may be selected from one or more of the group consisting of poly(L,D-lactic-co-glycolic acid) (PLGA), poly(L-lactide) (PLLA), poly(ε-caprolactone) (PCL), poly(glycolide) (PGA), poly(lactide) (PLA), and co-polymers thereof (e.g. the hydrophobic polymer may be a blend of PLLA and PCL, optionally wherein the PLLA and PCL form a blend having a w/w ratio of from 5:1 to 1:5, such as 3:1);
(B) the osmotic agent may be substantially distributed in the hollow core of the hollow core-shell microcapsule;
(C) the flotation agent may be substantially distributed in the hollow core of the hollow core-shell microcapsule;
(D) the ratio of hydrophilic or amphiphilic carrier matrix to the hydrophobic polymer may be from 1:100 to 1:3 w/w, such as from 1:50 to 1:8 w/w, such as from 1:40 to 1:10 w/w;
(E) the hydrophilic or amphiphilic carrier matrix may be selected from one or more of the group consisting of alginate, chitosan, casein, starch, hyaluronic acid, gelatin, agarose, collagen, fibrin, dextran, polyvinylalcohol (PVA) and polyethylene glycol (PEG), optionally wherein the hydrophilic or amphiphilic carrier matrix may be casein;
(F) the hydrophilic or amphiphilic carrier matrix may be an amphiphilic carrier matrix;
(G) the flotation agent may be an oil (e.g. the oil may be selected from one or more of the group consisting of fish oil, olive oil, corn oil, sunflower seed oil, grape seed oil, canola oil, avocado oil, and coconut oil, optionally wherein the flotation agent may be selected from fish oil and/or olive oil);
(H) the flotation agent may be present in an average amount of from 0.1 to 10 wt % of the total weight of the microcapsule;
(I) the microcapsule may be capable of floating in a simulated digestive fluid for a period of from 48 to 72 hours;
(J) the microcapsule may have an average diameter of from 100 to 1,200 μm, such as from 200 to 1,000 μm, such as from 500 to 800 μm, such as 600 μm;
(K) the first drug may be a hydrophilic drug;
(L) the osmotic agent may be an alkaline metal salt or an alkaline earth salt, such as sodium chloride;
(M) the microcapsule may further comprise a second drug that is hydrophobic and distributed within the hydrophobic polymer shell;
(N) the microcapsule may be suitable for use to treat a chronic condition (e.g. the chronic condition is selected from one or more of the group consisting of Parkinson's disease, diabetes, tuberculosis, stroke, HIV, mental disorders, cancer, Alzheimer's disease, disorders of lipid metabolism, lupus, metabolic syndrome, hypertension, chronic renal failure, inflammation, obesity, atherosclerosis, angina pectoris, myocardial infarction, gastric ulcer, alcoholic liver disease, and degenerative arthritis, in particular embodiments, the chronic condition is selected from the group consisting of Parkinson's disease, diabetes, and tuberculosis.

In embodiments of the invention where there is a first (hydrophilic) and a second (hydrophobic) drug, where the first drug is distributed within the hydrophilic or amphiphilic carrier matrix and the second drug is distributed within the hydrophobic polymer shell:

(aa) the first drug comprises levodopa (LD) and carbidopa (CD) and the second drug comprises entacapone (ENT);
(ba) the first drug comprises metformin (MET) and the second drug comprises fenofibrate (FEN); or
(ca) the first drug comprises isoniazid (ISO) and ethambutol (ETH) and the second drug comprises rifampicin (RIF)

In a second aspect of the invention, there is provided a method of forming a sustained release hollow core-shell microcapsule formulation for drug delivery as defined in the first aspect of the invention and any one of its embodiments, comprising the steps of:
(a) providing a water₁/oil emulsion, where
   the water₁ phase comprises a hydrophilic or amphiphilic carrier matrix material, a first drug and an osmotic agent,
   the oil phase comprises an organic solvent, one or more hydrophobic polymers and a flotation agent;
(b) adding the water₁/oil emulsion to an aqueous solution having a first volume and a pH value of from 2 to 6 and agitating at ambient temperature for a period of time to form a water₁/oil/water₂ emulsion;
(c) adding a second volume of an aqueous solution having a pH value of from 2 to 6 to the water₁/oil/water₂ emulsion to form a final intermediate mixture; and
(d) subjecting the final intermediate mixture to a centrifugal force and removing the organic solvent and, optionally, the water under reduced pressure to form the hollow core-shell microcapsules.

In embodiments of the second aspect of the invention:

(i) the concentration of the hydrophilic or amphiphilic carrier matrix material in the water₁ phase may be from 1 mg/mL to 100 mg/mL, such as from 5 mg/mL to 75 mg/mL, such as from 10 to 50 mg/mL;
(ii) the concentration of the osmolyte in the water₁ phase may be from 0.1 mg/mL to 10 mg/mL, such as from 0.5 mg/mL to 5 mg/mL, such as from 1 to 2 mg/mL;
(iii) the concentration of the flotation agent in the oil phase may be from 0.01 to 2% v/v, such as from 0.05 to 1% v/v, such as from 0.1 to 0.3% v/v, such as from 0.15 to 0.2% v/v; and/or
(iv) the agitation in step (b) may be provided by a stirrer operating at from 50 to 2,000 rpm, such as from 100 to 1,500 rpm, such as from 200 to 1,000 rpm, such as from 300 to 750 rpm, such as 400 to 600 rpm;
(v) the pH of the water₂ phase may be from 3 to 5, such as 4;
(vi) the aqueous solution of the water₂ phase may comprise PVA in a concentration to provide an aqueous solution having a pH value of from 2 to 6, such as from 3 to 5, such as 4; and/or
(vii) the water₂ phase may further comprise an amount of the organic solvent greater than or equal to the solubility of said organic solvent in water;
(viii) the total volume to volume ratio of the organic solvent to the water₂ phase may be from 3 to 50% v/v, such as from 5 to 25% v/v, such as from 12 to 20% v/v, such as 15% v/v;
(ix) the organic solvent may be selected from one or more of the group consisting of dichloromethane, chloroform, toluene, pentane, hexane, heptane, octane, nonane, n-decane, n-dodecane, benzyl chloride, hexadecane, diethyl ether, ethyl acetate, cyclohexane, chloromethane, trichloroethylene (TCE), benzene, bromodichloromethane, vinyl chloride, trichloroethane, methyl ethyl ketone, methyl isobutyl ketone, methyl tert-butyl ether, vinyl acetate, dichloroethane, chloroethane, trichlorotrifluoroethane, ethylbenzene and isopropylbenzene, optionally wherein the organic solvent is dichloromethane;
(x) in step (c) of the method the volume to volume ratio of the second aqueous solution to first aqueous solution may be from 1:1 to 10:1, such as from 2:1 to 5:1, such as 3:1;
(xi) in step (c) of the method the second aqueous solution may comprise PVA in a concentration to provide an aqueous solution having a pH value of from 2 to 6, such as from 3 to 5, such as 4;
(xii) the hydrophobic polymer may be selected from one or more of the group consisting of poly(L,D-lactic-co-glycolic acid) (PLGA), poly(L-lactide) (PLLA), poly(ε-caprolactone) (PCL), poly(glycolide) (PGA), poly(lactide) (PLA), and co-polymers thereof, optionally wherein the hydrophobic polymer may be a blend of PLLA and PCL (e.g. the hydrophobic polymer may be a blend of PLLA and PCL having a PLLA/PCL w/w ratio of from 5:1 to 1:5, such as 3:1);
(xiii) the ratio of hydrophilic or amphiphilic carrier matrix material to the hydrophobic polymer may be from 1:100 to 1:3 w/w, such as from 1:50 to 1:8 w/w, such as from 1:40 to 1:10 w/w;
(xiv) the hydrophilic or amphiphilic carrier matrix material may be an amphiphilic carrier matrix material or the hydrophilic or amphiphilic carrier matrix material is selected from one or more of the group consisting of alginate, chitosan, casein, starch, hyaluronic acid, gelatin, agarose, collagen, fibrin, dextran, polyvinylalcohol (PVA) and polyethylene glycol (PEG), optionally wherein the hydrophilic or amphiphilic carrier matrix material may be casein;
(xv) the flotation agent may be an oil, optionally wherein the oil may be selected from one or more of the group consisting of fish oil, olive oil, corn oil, sunflower seed oil, grape seed oil, canola oil, avocado oil, and coconut oil, for example, the flotation agent may be selected from fish oil and/or olive oil;
(xvi) the resulting microcapsule may have an average diameter of from 100 to 1,200 μm, such as from 200 to 1,000 μm, such as from 500 to 800 μm, such as 600 μm;
(xvii) the osmotic agent may be an alkaline metal salt or an alkaline earth salt, such as sodium chloride;
(xviiii) the first drug may be a hydrophilic drug;
(xix) the oil phase of the water₁/oil emulsion may further comprise a second drug that is hydrophobic.

In embodiments of the second aspect of the invention, where the first (hydrophilic) drug is in the water₁ phase and the second (hydrophobic) drug is in the oil phase of the water₁/oil emulsion:

(ab) the first drug comprises levodopa (LD) and carbidopa (CD) and the second drug comprises entacapone (ENT);
(bb) the first drug comprises metformin (MET) and the second drug comprises fenofibrate (FEN); or
(cb) the first drug comprises isoniazid (ISO) and ethambutol (ETH) and the second drug comprises rifampicin (RIF).

In a third aspect of the invention, there is provided a use of a sustained release hollow core-shell microcapsule formulation as described in the first aspect of the invention, and any technically sensible combination of its embodiments, for sustained drug(s) release in the gastrointestinal tract, optionally wherein the release of the drug(s) occurs predominantly in the stomach.

In a fourth aspect of the invention, there is provided a method of treating a chronic disease, comprising the step of administering a suitable amount of a sustained release hollow core-shell microcapsule formulation as described in the first aspect of the invention, and any technically sensible combination of its embodiments, to a subject in need thereof.

In a fifth aspect of the invention, there is provided a use of a sustained release hollow core-shell microcapsule formulation as described in the first aspect of the invention, and any technically sensible combination of its embodiments, in the preparation of a medicament to treat a chronic disease.

In a sixth aspect of the invention, there is provided a sustained release hollow core-shell microcapsule formulation as described in the first aspect of the invention, and any technically sensible combination of its embodiments, for use in the treatment of a chronic disease.

In embodiments of the third to sixth aspects of the invention, the chronic disease is selected from one or more of the group consisting of Parkinson's disease, diabetes, tuberculosis, stroke, HIV, mental disorders, cancer, Alzheimer's disease, disorders of lipid metabolism, lupus, metabolic syndrome, hypertension, chronic renal failure, inflammation, lupus, obesity, atherosclerosis, angina pectoris, myocardial infarction, gastric ulcer, alcoholic liver disease, and degenerative arthritis.

### DRAWINGS

Certain embodiments of the present disclosure are described more fully hereinafter with reference to the accompanying drawings.

FIG. 1. SEM images of cross-sectioned (a) 1%, (b) 3%, and (c) 5% (w/v) of casein/PLLA+PCL microcapsules loaded with LD, CD and ENT and salt (2 mg) in simulated gastric fluid (SGF) after 0 (Left column), 24 (Middle column), and 48 h (Right column). Row 1: sample F1; Row 2: sample F2; Row 3: sample F3 (please refer to Table 1 for a description for each sample).

FIG. 2. Buoyancy (%) of the different microcapsules encapsulating drugs (samples F1, F2, F3) in SGF at 37° C. for 48 h (n=3).

FIG. 3. Raman spectra depicting a casein-containing hollow core and a PLLA/PCL shell from sample F2 (3% (w/v) casein-loaded microcapsule).

FIG. 4. Release profiles of levodopa (LD), carbidopa (CD), and entacapone (ENT) from a) Control I microcapsules (0%), b) F1 (1%), c) F2 (3%), and d) F3 (5% (w/v) casein/PLLA+PCL microcapsules) in SGF at 37° C. for 48 h (n=5).

FIG. 5. Release profiles of metformin (MET) and fenofibrate (FEN) from a) Control II microcapsules (0%), b) F4 (1%), c) F5 (3%), and d) F6 (5% (w/v) casein/PLLA+PCL microcapsules) in SGF at 37° C. for 48 h (n=5).

FIG. 6 (appears to correspond to FIG. 2 of the additional results provided). In vitro release profiles of three tuberculosis drugs (hydrophilic Isoniazid (ISO), Ethambutol (ETH) and hydrophobic Rifampicin (RIF)) from a) Control III microcapules (0%), b) F7 (1%), c) F8 (3%), and d) F9 (5% (w/v) casein/PLLA+PCL microcapsules) in SGF at 37° C. for 48 h (n=5).

FIG. 7. Plots showing (a) water uptake and (b) change in molecular weight of PLLA of the degrading F1 (1%), F2 (3%) and F3 (5% (w/v) casein/PLLA+PCL microcapsules), as a function of incubation time (n=5).

FIG. 8 (FIG. 1 of additional results). In vitro release profiles of three tuberculosis drugs (hydrophilic Isoniazid (ISO), Ethambutol (ETH) and hydrophobic Rifampicin (RIF)) from different casein-microcapsules (casein concentration: 0, 1, 3, 5% (w/v)) in SGF for 4 h, followed by SIF for 48 h at 37° C. (n=5).

FIG. 9. Plasma concentrations of (a) levodopa, (b) carbidopa, and (c) entacapone after oral administration of control solution and casein-microcapsule corresponding to levodopa/carbidopa/entacapone (10/2.5/20 mg/kg). Results are in terms of mean±SD (n=5).

FIG. 10. Normalized brain concentrations of (a) levodopa and (b) dopamine after oral administration of control solution and casein-microcapsule corresponding to levodopa/carbidopa/entacapone (10/2.5/20 mg/kg). Results are means±SD (n=5).

FIG. 11. (FIG. 7 of priority) In vitro release profiles of LD, CD, and ENT from a) Control I microcapsules (0%), b) F1 (1%), (c) F2 (3%), and (d) F3 (5% (w/v) casein/PLLA+PCL microcapsules) in SGF for 5 h followed by release into SIF at 37° C. for 48 h (n=5).

FIG. 12. (FIG. 8 of priority) In vitro release profiles of MET and FEN from a) Control II microcapsules (0%), b) F4 (1%), (c) F5 (3%), and (d) F6 (5% (w/v) casein/PLLA+PCL microcapsules) in SGF for 5 h followed by release into SIF at 37° C. for 48 h (n=5).

### DESCRIPTION

This invention relates to a method of preparing oral-administrable microcapsules for controlled and sustained release of encapsulated agents, and also relates to said microcapsules per se. More particularly, this invention relates to gastric-floating, hollow microcapsules that are designed to entrap multiple drugs at high drug loading efficiencies and with controlled release capabilities, and methods to fabricate the same. With such a delivery system, the aim is to reduce dosing frequency and pill burden, thus improving patient medication compliance. The examples section below shows how this delivery system can be used to release two or more (e.g. three) different drugs used in the management of tuberculosis, Type II diabetes and, more particularly Parkinson's disease. Examples of treatment of other diseases that would benefit from such a sustained-release drug delivery system include Alzheimer's disease, mental disorders, stroke, HIV, and lupus, all of which require multiple drug combination therapies.

As mentioned above, the disclosed invention relates to the microcapsules per se. Thus, there is disclosed a sustained release hollow core-shell microcapsule formulation for drug delivery, comprising:
a hollow shell having an outer surface and an inner surface that is formed from one or more hydrophobic polymers;
a hydrophilic or amphiphilic carrier matrix distributed over the inner surface of the hollow shell;
a first drug distributed within the hydrophilic or amphiphilic carrier matrix;
optionally, an osmotic agent; and
a flotation agent, wherein
the microcapsule is capable of floating in a simulated digestive fluid for a period of from 24 to 96 hours.

For the avoidance of any doubt, only the osmotic agent is an optional feature, for the reasons described in more detail below. All other components are required to be present in the microcapsules.

The above formulation provides a floating carrier formulation that captures both hydrophilic and hydrophobic drugs in respective suitable compartments (the carrier matrix and the hollow shell, respectively), thereby enabling controlled and sustained release of the hydrophilic and hydrophobic encapsulated drugs in the desired amount and over a desired period of time. The aim of this formulation is to reduce the required dosing of the (one or, more particularly, two or more) drug(s) to just once a day, or possibly even less frequently.

As will be appreciated, the formulation is intended for the oral delivery of one or more drugs and this oral delivery system has the following characteristics:

1. encapsulation of one or more different drugs (e.g. up to three or more different drugs);
2. excellent floatability, of up to 96 h (e.g. from 24 to 96 h, up to 48 h or from 48 to 72 hours) in a simulated digestive fluid (e.g. a simulated gastric fluid (SGF));
3. controlled and sustained release of the (multiple) encapsulated drug(s);
4. the formulation may be easily produced through a few simple steps; and
5. the formulation may have enhanced drug loading efficiencies.

In addition to the above, when there is more than one drug, the formulation described herein may enable each of these drugs to be released at a suitable rate to obtain the desired overall concentration steady state window in a subject for each drug.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

When used herein, the term “hollow core-shell microcapsule” relates to a microcapsule having a hollow section at its core, where the hollow core may contain one or more components, whether attached/coated to and distributed over the inner surface of the shell or freely-moving in the core. In other words, the hollow-core-shell microcapsule may resemble a bird's egg, where components attached/coated to and distributed over the inner surface of the shell may be loosely analogous to a membrane in an egg, while freely-moving components may be loosely analogous to the albumin of an egg. As will be appreciated, the analogy to a bird's egg is simply intended to enhance understanding of where and how the components are situated and is not intended to require the components described to function or to look anything like the described analogous component in a bird's egg.

As will be appreciated, the “outer surface” of the hollow shell refers to the surface of the hollow shell that is directly exposed to the external environment, while the “inner surface” of the hollow shell is the surface that defines the hollow space at the core of the shell.

The term “hydrophilic” is generally understood to describe a substance that has a high affinity for water. For example, a hydrophilic material may be one that is able to be dissolved in, be mixed with, be wetted by or absorbs water. In line with this definition, the term “hydrophilic polymer” has a high affinity for aqueous solutions.

The term “hydrophobic” is generally understood to describe a substance that repels water. For example, a hydrophobic material may include materials that do not dissolve in, be mixed with, be wetted by water or absorb an appreciable amount of water. In line with this definition, the term “hydrophobic polymer” refers to a polymer having a low affinity for aqueous solutions.

The term “amphiphilic” when used herein refers to a material that displays both hydrophilic and hydrophobic properties. Typically such materials must have at least two regions—one that is hydrophilic and one that is hydrophobic, but may have more than one region of each type. Typical amphiphilic compounds include materials such as fatty acids and lipoproteins, as well as copolymers (i.e. block copolymers) having blocks that carry hydrophilic and hydrophobic groups.

The term “drug” when used herein may refer to a substance useful for the treatment of or the prevention of a condition affecting a human or other animal. Said condition may be a disease, a disorder or a physiological condition. It will be appreciated that the drug may not directly affect the underlying condition, but may be used as an adjuvant with a further drug to enhance the effectiveness of the other drug. Thus, the term “drug” herein includes all classes of active agents, whether adjuvant or therapeutic, that may be provided to a subject through oral administration. In certain embodiments, the term “drug” may also be used herein with reference to nutraceuticals, cosmeceuticals and food-based nutrients, as discussed in more detail below.

The term “hydrophobic polymer” refers to a polymer having a low affinity for aqueous solutions including water. For example, hydrophobic polymers may include polymers that do not dissolve in, be mixed with, or be wetted by water. As another example, hydrophobic polymers may also include polymers that do not absorb an appreciable amount of water.

The hydrophobic polymer used in the present invention may be a natural polymer or a synthetic polymer. The term “natural polymer” as used herein refers generally to a polymeric material that may be found in nature. Examples of a natural hydrophobic polymer include, but are not limited to, natural rubber and alkylated celluloses, such as ethyl cellulose.

Examples of synthetic hydrophobic polymers include, but are not limited to, polyolefin, polystyrene, polyester, polyamide, polyether, polysulfone, polycarbonate, polyurea, polyurethane, polysiloxane, copolymers thereof, and blends thereof.

As will be appreciated, as the current invention relates to formulations for oral administration to living organisms, the hydrophobic polymer is preferably biocompatible. That is, the hydrophobic polymer is preferably a material that does not cause adverse side-effects in a subject following administration (e.g. capable of interacting with a biological system without causing cytotoxicity, undesired protein or nucleic acid modification or activation of an undesired immune response) and, more preferably, it is a material that can be degraded in vivo (e.g. hours, days, months or years). Disintegration may for instance occur via hydrolysis, may be catalyzed by an enzyme and may be assisted by conditions to which the microparticles are exposed to in vivo. Examples of such suitable hydrophobic polymers include, but are not limited to, oligomers of glycolide, lactide, polylactic acid, polyesters of α-hydroxy acids, including lactic acid and glycolic acid, such as the poly(α-hydroxy) acids including polyglycolic acid, poly(D,L-lactic-co-glycolic acid) (PLGA), poly-L-lactic acid (PLLA), and terpolymers of D,L-lactide and glycolide; ε-caprolactone and ε-caprolactone copolymerized with polyesters; polylactones and polycaprolactones including poly(caprolactone) (PCL), poly(ε-caprolactone), poly(valerolactone) and poly(gamma-butyrolactone); polyanhydrides; polyorthoesters; polydioxanone; and other biologically degradable polymers that are nontoxic or are present as metabolites in the body. Further suitable hydrophobic polymers include, but are not limited to, polyamides, polycarbonates, polyalkylenes, polyalkylene glycols, polyalkylene oxides, polyalkylene terepthalates, polyvinyl ethers, polyvinyl esters, polyvinyl halides, polyvinylpyrrolidone, polyglycolides, polysiloxanes, polyurethanes and copolymers thereof, nitro celluloses, polymers of acrylic and methacrylic esters, ethyl cellulose, cellulose acetate, cellulose propionate, cellulose acetate butyrate, cellulose acetate phthalate, carboxylethyl cellulose, cellulose triacetate, poly(methylmethacrylate), poly(ethylmethacrylate), poly(butylmethacrylate), poly(isobutylmethacrylate), poly(hexylmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene, poly(ethylene terephthalate), such as ethylene vinyl acetate (EVA), polyvinyl chloride, polystyrene, gluten, polyanhydrides, any copolymers thereof, and mixtures thereof.

As will be appreciated, a single hydrophobic polymer or a multiple (i.e. 2, 3, 4, or 5) hydrophobic polymer blend may be used to form the capsule shell. For a blend of multiple polymers, any suitable ratio can be used, depending on the desired properties to be obtained by the shell, which may be readily determined by a person skilled in the art of such formulation techniques. As will be appreciated, differences in physicochemical properties between two or more polymers may generate pores due to the immiscibility of said polymers. For example, where there are two polymers, one may have a higher solubility than the other, resulting in a porous structure. In addition, the use of the osmotic agents in the methods of manufacture disclosed herein may also allow the influx of water into the core, and in the process create pores in the shell.

In particular embodiments of the invention, the hydrophobic polymer may be selected from one or more of the group consisting of poly(L,D-lactic-co-glycolic acid) (PLGA), poly(L-lactide) (PLLA), poly(ε-caprolactone) (PCL), poly(glycolide) (PGA), poly(lactide) (PLA), and co-polymers thereof. For example, the hydrophobic polymer may be a blend of PLLA and PCL. In examples of such blends, the w/w ratio of PLLA to PCL may be from 5:1 to 1:5, such as 3:1.

In general embodiments of the invention, the hydrophobic polymer may make up from 50 to 95 wt % of the microcapsule formulation on average. For example, in further general embodiments of the invention, the hydrophobic polymer may make up from 60 to 90 wt % of the microcapsule formulation on average, such as from 75 to 89 wt % on average.

The hydrophilic carrier matrix is a hydrophilic polymer that provides a polymeric matrix that may encapsulate one or more drugs (e.g. hydrophilic drugs). Hydrophilic polymers that may be mentioned herein include, but are not limited to polyamines having amine groups on either the polymer backbone or the polymer side chains, polyvinyl alcohol (PVA), polyethylene glycol (PEG), naturally occurring proteins, poly(oxyalkylene oxides), polysaccharides and polysaccharide derivatives, complex sugars and polyacrylamides.

Examples of polyamines having amine groups on either the polymer backbone or the polymer side chains include, but are not limited to poly-L-lysine and other positively charged polyamino acids of natural or synthetic amino acids or mixtures of amino acids, including poly(D-lysine), poly(ornithine), poly(arginine), and poly(histidine), and nonpeptide polyamines such as poly(aminostyrene), poly(aminoacrylate), poly(N-methyl aminoacrylate), poly(N-ethylaminoacrylate), poly(N, N-dimethylaminoacrylate), poly(N, N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylaminopropyltrimethyl ammonium chloride), poly(ethyloxazoline), poly(N-vinyl pyrrolidone), and neutral poly(amino acids) such as poly(serine), poly(threonine), and poly(glutamine). Examples of naturally occurring proteins include, but are not limited to gelatin, bovine serum albumin, and ovalbumin. Examples of complex sugars include, but are not limited to hyaluronic acid, starches and agarose. Examples of poly(oxyalkylene oxides) include, but are not limited to, poly(ethylene oxide) and poly(vinyl alcohol). Examples of natural or synthetic polysaccharides and polysaccharide derivatives include, but are not limited to, alginate, chitosan, dextran, and water soluble cellulose derivatives such as hydroxy ethyl cellulose and carboxymethylcellulose. As used herein, “derivatives” include polymers having substitutions, additions of chemical groups, for example, alkyl, alkylene, hydroxylations, oxidations and other modifications routinely made by those skilled in the art. Examples of polyacrylamides include, but are not limited to poly(hydroxyethyl acrylate), poly(hydroxy ethylmethacrylate), and isopropylacrylamide. The hydrophilic polymer can also be any biocompatible water-soluble polyelectrolyte polymer. In one embodiment, a polycationic polymer, for example, any polymer having protonated heterocycles attached as pendant groups, can be utilised. Particular hydrophilic polymers that may be mentioned in embodiments of the invention include, but are not limited to, alginate, chitosan, starch, hyaluronic acid, gelatin, agarose, collagen, fibrin, dextran, polyvinylalcohol (PVA) and polyethylene glycol (PEG).

The amphiphilic carrier matrix is an amphiphilic polymer that provides a polymeric matrix that may encapsulate one or more drugs (e.g. hydrophilic drugs). Amphiphilic polymers that may be disclosed herein include casein, random or more preferably block copolymers of compatible hydrophilic and hydrophobic polymers (e.g. from the lists disclosed above) and derivatives of hydrophilic polymers that have been subjected to substantial alkylation (e.g. with C₁ to C₅₀ alkyl linear or branched chains) of polar side groups capable of being so functionalised. A particular amphiphilic polymer that may be mentioned in embodiments of the current invention is casein.

As will be appreciated, a single hydrophilic or amphiphilic polymer may be used in the formulations described herein to form the carrier matrix. Alternatively a blend comprising multiple (e.g. 2, 3, 4 or 5) hydrophilic and/or amphiphilic polymers may also be used to form the carrier matrix. For such blends any suitable ratio of the constituent hydrophilic and/or amphiphilic polymers may be used, depending on the desired properties of the carrier matrix, which may be readily determined by a person skilled in the art of such formulation techniques.

As will be appreciated, the amount of carrier matrix provided to the hollow core shell microcapsules may be any suitable amount to obtain the desired effect, With that in mind, the ratio of the hydrophilic or amphiphilic carrier matrix to the hydrophobic polymer may be from 1:100 to 1:3 w/w, such as from 1:50 to 1:8 w/w, such as from 1:40 to 1:10 w/w. For example, in certain embodiments, the hydrophilic or amphiphilic carrier matrix may be present in an average amount of from 1 to 20 wt % of the total weight of the capsule, while the hydrophobic polymer may be present in an amount of from 75 to 90 wt % of the total weight of the capsule. In certain embodiments, the hydrophilic or amphiphilic carrier matrix may be present in an average amount of from 2 to 11 wt % of the total weight of the capsule, while the hydrophobic polymer may be present in an amount of from 79 to 89 wt % of the total weight of the capsule.

As will be appreciated, the various weight percentage values of the components cited in relation to the microcapsules herein may be combined in every possible technically sensible combination.

Without wishing to be bound by theory, it is believed that the hydrophilic or amphiphilic carrier matrix swells upon contact with water that diffuses through the shell, which water then enables the diffusion of the first drug (e.g. one or more hydrophilic drugs) out of the hollow core, through the shell and into the gastrointestinal tract.

In particular embodiments of the invention the hydrophilic or amphiphilic carrier matrix may be casein. Particular advantages associated with the use of casein as the carrier matrix include the ability to alter its physical properties through adjusting the pH of the surrounding environment, and its nutritional value as a food protein. In addition, casein has several advantages such as cost-effectiveness, non-toxicity and good biodegradability. Casein also has a good affinity with small molecules, as the casein structure consists of hydrophobic and hydrophilic domains. This unique structure allows casein to encapsulate both hydrophobic and hydrophilic drugs for sustained drug release.

When used herein, the term “osmotic agent” refers generally to compounds or substances that affect osmosis. The osmotic agent is used in the process to manufacture the microcapsules and acts to draw in water to harden/precipitate the hydrophilic or amphiphilic carrier matrix. As such, the osmotic agent may or may not be present in the microcapsules. When present in the microcapsules, the osmotic agent may be substantially distributed in the hollow core of the hollow core-shell microcapsule. By “substantially distributed in the hollow core”, we mean that the majority (i.e. greater than 50%, greater than 60%, greater than 70%, greater than 80%, greater than 90%, such as from 95 to greater than 99.99%) of the osmotic agent remaining in the formed microcapsules may be located within the hollow core portion of the microcapsule. That is, the osmotic agent may be in the form of discrete freely-moving particles within the hollow core or it may be partly or wholly encapsulated in the carrier matrix (e.g. it is freely-moving particles). For any osmotic agent that is not within the hollow core, then it is wholly or partly encapsulated by the hydrophobic polymer of the shell. The osmotic agent may be an alkaline metal salt or an alkaline earth salt. Examples of osmotic agents that may be used herein include, but are not limited to, sodium chloride, potassium chloride, sodium bromide, sodium citrate, sodium lactate, sodium hydroxide, sodium iodide, sodium carbonate, sodium hydrogen carbonate, sodium nitrate, sodium fluoride, sodium sulfate, potassium carbonate, potassium citrate, potassium lactate, potassium hydrogen carbonate, potassium bromide, potassium hydroxide, potassium iodide, potassium nitrate, potassium sulfate, cesium chloride, rubidium chloride, lithium chloride, and mixtures thereof. In various embodiments, the osmotic agent may comprise or consist essentially of sodium chloride.

When used herein, the flotation agent may be anything that has a lower density than water (i.e. the flotation agent is a material that has a density of less than 1 g/mL at 25° C.). An example of a suitable class of materials for use as a flotation agent in the current invention is oil. Suitable oils that may be mentioned herein include, but are not limited to fish oil, olive oil, corn oil, sunflower seed oil, grape seed oil, canola oil, avocado oil, and coconut oil. In particular embodiments that may be mentioned herein, the flotation agent may be fish oil and/or olive oil. The flotation agent may be substantially located in the hollow shell of the microcapsules. The flotation agent may be present in the capsules in any suitable amount, provided that it provides some buoyancy to the microcapsule. Such suitable amounts may include an average amount of from 0.1 to 10 wt % of the total weight of the microcapsule (e.g. for an oil).

When used herein “simulated digestive fluid” refers to a fluid that provides a pH environment similar to that of a part of the gastrointestinal tract. As an example simulated gastric fluid may have a pH value of from 1 to 3.5 (e.g. 1 or 1.5) to simulate that pH of the stomach, simulated intestinal fluid may have a pH value of from 2 to 9, depending on which part of the intestinal system is being simulated (e.g. the duodenum has a pH range of from 2 to 6, the jujenum has a pH range of from 7 to 9 and the ileum has a pH range of from 7 to 8). In embodiments that may be mentioned herein the simulated digestive fluid may be a simulated intestinal fluid having a pH value of from 6.5 to 7.4 or, more particularly, a simulated gastric fluid having a pH value of about 1, such as from 1 to 1.5.

As noted above, the microcapsule is capable of floating in a simulated digestive fluid for a period of from 24 to 96 hours. As will be appreciated, the desired floating time may be from 24 hours to 48 hours or from 48 hours to 96 hours, such as from 24 hours to 72 hours, such as from 48 to 72 hours or from 72 hours to 96 hours.

Generically, such a product may be described as microcapsules that comprise a casein-loaded (or other similar material) core; a polymer shell; hydrophilic drugs encapsulated in the casein-loaded core; and hydrophobic drugs encapsulated in the polymer shell.

As will be appreciated, the microcapsules may be any suitable size that provides the desired sustained release profile for the drug(s) within the capsules. Suitable sizes (i.e. diameters) for the microcapsules that may be mentioned herein include, but are not limited to an average diameter of from 100 to 1,200 μm, such as from 200 to 1,000 μm, such as from 500 to 800 μm, such as 600 μm.

As intimated herein the first drug may be a hydrophilic drug. Hydrophilic drugs are compounds that are polar that may have a partition coefficient log P in the range of from −5.0 to +1.0 (e.g. from −5.6 to +0.75). As will be appreciated, the term “first drug” may refer to one or more (e.g. 2, 3, 4) active ingredients, whether therapeutic or adjuvant in nature. In certain embodiments of the invention, the microcapsule may further comprise a second drug that is hydrophobic and distributed within the hydrophobic polymer shell. Hydrophobic drugs are compounds that are lipophilic in nature and may display a partition coefficient log Pin the range of from +1.0 to +10 (e.g. from +1.25 to +10). As will be appreciated, the term “second drug” may refer to one or more (e.g. 2, 3, 4) active ingredients, whether therapeutic or adjuvant in nature.

Oral administration is still considered the preferred route for administrating therapeutic agents because of its low cost, ease of administration and high level of patient compliance. Thus, the microcapsules disclosed herein are particularly suited to this route of administration—not least because they are designed to float in the stomach and other compartments of the digestive tract (e.g. an enterically coated capsule containing the microcapsules may pass through the stomach and reach the duodenum/jujenum/ileum before releasing the microcapsules into the digestive fluids, whereupon they may float in the compartment of release). As for the treatment and management of chronic diseases, multiple drugs which have different hydrophilicities (i.e. hydrophobic or hydrophilic) are usually required and administrated by oral route. The current invention provides a system that allows for co-encapsulation and controlled release of multiple drugs from a floating core/shell structured microcapsule with an enhanced drug loading efficiency, prolonged gastric residence time and sustained/controlled release capability.

Example embodiments of the formulations described herein may one or more hydrophilic drugs and one or more hydrophobic drugs dispersed within the carrier matrix or the shell, respectively. Such combinations may include:

(aa) the hydrophilic drugs levodopa (LD) and carbidopa (CD), and the hydrophobic drug entacapone (ENT);
(ba) the hydrophilic drug metformin (MET) and the hydrophobic drug fenofibrate (FEN); or
(ca) the hydrophilic drugs isoniazid (ISO) and ethambutol (ETH) and the hydrophobic drug rifampicin (RIF).

Examples of microcapsules disclosed herein may comprise: a casein-loaded core; a polymer shell; hydrophilic drugs encapsulated in the casein-loaded core; and hydrophobic drugs encapsulated in the polymer shell.

As noted hereinbefore, the formulations herein may be particularly suited to assisting in the management/treatment of chronic conditions in a subject. Thus, the invention also relates to:

(AA) a use of a sustained release hollow core-shell microcapsule formulation as described hereinbefore, and any technically sensible combination of its embodiments, for sustained drug(s) release in the gastrointestinal tract, optionally wherein the release of the drug(s) occurs predominantly in the stomach;
(AB) a method of treating a chronic disease, comprising the step of administering a suitable amount of a sustained release hollow core-shell microcapsule formulation as described hereinbefore, to a subject in need thereof;
(AC) a use of a sustained release hollow core-shell microcapsule formulation as described in the first aspect of the invention as described hereinbefore in the preparation of a medicament to treat a chronic disease; and
(AD) a sustained release hollow core-shell microcapsule formulation as described hereinbefore, for use in the treatment of a chronic disease.

Examples of chronic conditions and/or diseases (which may be used herein interchangeably) include, but is not limited to Parkinson's disease, diabetes, tuberculosis, stroke, HIV, mental disorders, cancer, Alzheimer's disease, disorders of lipid metabolism, lupus, metabolic syndrome, hypertension, chronic renal failure, inflammation, obesity, atherosclerosis, angina pectoris, myocardial infarction, gastric ulcer, alcoholic liver disease, and degenerative arthritis. Particular chronic conditions that may be mentioned herein include tuberculosis or, more particularly, diabetes (e.g. type II diabetes) or, yet more particularly, Parkinson's disease.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, came, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The terms “effective amount” and “suitable amount” and variants thereof refer to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

As noted hereinbefore, the microcapsules are intended for oral administration to a subject for effecting treatment of said subject. As such, the formulation may further comprise a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995).

Otherwise, the preparation of suitable formulations may be achieved routinely by the skilled person using routine techniques and/or in accordance with standard and/or accepted pharmaceutical practice. For example, the microcapsules described herein may be packaged in a tablet or, more particularly, a capsule (e.g. a gelatin capsule) for ease of administration, where said tablet/capsule is selected to release the microcapsules shortly after delivery to the stomach.

The amount of the drug(s) in any pharmaceutical formulation used in accordance with the present invention will depend on various factors, such as the severity of the condition to be treated, the particular patient to be treated, as well as the compound(s) which is/are employed. In any event, the amount of compound in the formulation may be determined routinely by the skilled person.

For example, a solid oral composition such as a tablet or capsule may contain from 0.5 to 20% (w/w) active ingredient(s) in the microcapsules; from 50 to 99% (w/w) of the microcapsules (including the drug(s)), from 0 to 50% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment.

As will be appreciated, the amounts of each drug (whether hydrophobic or hydrophilic) included in each microcapsule will depend on the desired eventual dosage of the drug in question. For example, each drug (whether hydrophobic or hydrophilic) loaded into the microcapsules may be present in an average amount of from 0.001 wt % to 24 wt %, such as from 0.1 wt % to 15 wt %, such as from 0.5 wt % to 10 wt % (e.g. from 0.7 wt % to 6 wt %) of the total weight of the capsule. When the capsules contain more than one drug, the total weight percentage of all drugs in the microcapsule may be from 0.001 to 24 wt %, such as from 5 to 15 wt %, such as from 8 to 10 wt %.

Depending on the disorder, and the patient, to be treated, as well as the route of administration, the formulations may be administered at varying therapeutically effective doses to a patient in need thereof. However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

The dosage may also be determined by the timing and frequency of administration. In the case of oral or parenteral administration the dosage can vary from about 0.01 mg to about 1000 mg per day of each drug.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

As will be appreciated, this invention aims to reduce dosing for a chronic condition to just once a day, or possibly even less frequently. With that in mind, the oral microcapsules disclosed herein (i.e. the microcapsules of the invention) may provide controlled release for each drug contained therein, either at similar release rates or, if preferred, different release rates. For example, when gastric-floating, casein-loaded hollow microcapsules that co-encapsulate multiple PD drugs (i.e. levodopa (LD), carbidopa (CD) and entacapone (ENT)) are prepared and used, the oral microcapsules can provide controlled release of all three PD drugs at similar rates.

The aspects of the invention described herein (e.g. the above-mentioned formulation, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

As will be appreciated, the compositions disclosed herein may also be suitable for the delivery of nutraceuticals, cosmeceuticals and food-based nutrients, whether in the treatment of a chronic condition or otherwise. Thus, in certain embodiments of the invention, the terms “drug”, “first drug” and “second drug” may be applied to nutraceuticals, cosmeceuticals and food-based nutrients. In this context, the “first drug” will relate to hydrophilic nutraceuticals, cosmeceuticals and food-based nutrients, while the second drug will relate to hydrophobic nutraceuticals, cosmeceuticals and food-based nutrients.

“Nutraceutical” is a portmanteau of “nutritional” and “pharmaceutical” and refers to foods thought to have a beneficial effect on human health. It can also refer to individual chemicals which are present in common foods. Many such nutraceuticals are phytonutrients.

Nutraceuticals are sometimes called functional foods. Suitable nutraceuticals that may be mentioned herein may be:

(a) hydrophilic nutraceuticals which may be selected from, but not limited to, phenolic compounds (such as, for example, Resveratrol), Quercetin, Rutin, polyphenols (such as, for example, Oilgonol from lychee fruit), catechins, bioactive polysaccharides (such as, for example, Active Hexose Correlated Compound or AHCC), cofactors (such as, for example, pyrroloquinoline quinone (PQQ)), amino acids (such as, for example, arginine and glutamine), and mixtures thereof; and
(b) hydrophobic nutraceuticals which may be selected from, but not limited to, mixed carotenoids, carotenoid esters, Curcuminoids (e.g. Curcumin), Policosanol, Silymarin, Baicalein, Quercetin, plant sterols, vitamins (such as, for example, Vitamin E and A), alpha lipoic acid, sesquiterpene lactones (such as, for example, parthenolides), and mixtures thereof.

“Cosmeceutical” is also a hybrid term incorporating the concept of improving skin appearance by application of active ingredients that often serve a therapeutic role. Suitable cosmeceuticals may include, but are not limited to a desquamating agent, a moisturizer, a depigmenting or pro-pigmenting agent, an anti-glycation agent, an NO-synthase inhibitor, a 5α-reductase inhibitor, a lysyl and/or prolyl hydroxylase inhibitor, an agent for stimulating the synthesis of dermal or epidermal macromolecules and/or for preventing their degradation, an agent for stimulating the proliferation of fibroblasts and keratinocytes and/or keratinocyte differentiation, a muscle relaxant, a compound for reducing irritation, an antimicrobial agent, a tensioning agent, an anti-pollution agent, a free-radical scavenger and mixtures thereof.

“Food-based nutrient” refers to any material that may be beneficial to the human or animal body that may be obtained from a source of food. Such materials may include, but are not limited to, a vitamin such as vitamin A, B1, B2, B3, B6, B12, D, E, biotin, folate, and panothenate; minerals such as calcium, magnesium, selenium, and zinc; an amino acid such as asparagine, carnitine, glutamine, and serine; an antioxidant selected from coenzyme Q10, glutathione, and cysteine; or a metabolite such as lipoic acid, oleic add, choline, inositol, fructose, glucose, and insulin, and mixtures thereof.

As will be appreciated, the compositions and uses described herein may employ materials selected from the classes of standard active pharmaceutical ingredients (i.e. active therapeutic agents and/or adjuvants), nutraceuticals, cosmeceuticals and food-based nutrients. Any suitable combination of these classes may be used. For example, the material may solely contain active pharmaceutical ingredients or may contain one or more active pharmaceutical ingredients in combination with a nutraceutical and the like. In particular embodiments that are discussed herein, the compositions may comprise (or contain) active pharmaceutical ingredients only.

As will be appreciated, the microcapsules may be formed by a process that enables the rapid and convenient formation of microcapsules containing one or more drugs in a few steps through the formation of a water₁/oil/water₂ emulsion. Thus, there is provided a method of forming a sustained release hollow core-shell microcapsule formulation for drug delivery as defined above, comprising the steps of:
(a) providing a water₁/oil emulsion, where
   the water₁ phase comprises a hydrophilic or amphiphilic carrier matrix material, a first drug and an osmotic agent,
   the oil phase comprises an organic solvent, one or more hydrophobic polymers and a flotation agent;
(b) adding the water₁/oil emulsion to an aqueous solution having a first volume and a pH value of from 2 to 6 and agitating at ambient temperature for a period of time to form a water₁/oil/water₂ emulsion;
(c), adding a second volume of an aqueous solution having a pH value of from 2 to 6 to the water₁/oil/water₂ emulsion to form a final intermediate mixture; and
(d) subjecting the final intermediate mixture to a centrifugal force and removing the organic solvent and, optionally, the water under reduced pressure to form the hollow core-shell microcapsules.

As will be appreciated, further downstream processing steps may be conducted on the resulting microcapsules, such as freeze drying/lyophilisation and further steps to form a pharmaceutical formulation for oral delivery (e.g. as a liquid, a tablet or a capsule). Such steps may be accomplished using common knowledge of the person skilled in the art.

Conventional floating drug delivery systems involve monolithic systems whereby only a single drug is encapsulated, and these often lack controlled release capabilities. At the same time, the drug encapsulation efficiencies (in wt %) are often low. The process described above, enables the microcapsules formulated here to:

1. encapsulate one or more different drugs (e.g. up to three or more different drugs),
2. be easily produced through a few simple steps;
3. have enhanced drug loading efficiencies; and
4. prolonged gastric residence time and sustained/controlled release capability.

As will be appreciated, the components of the process that are the same as the components described above for the microcapsules maintain the same definitions provided hereinbefore unless explicitly stated otherwise. For example, the term “hydrophilic or amphiphilic carrier matrix material” refers to the “hydrophilic or amphiphilic carrier matrix” previously defined hereinbefore.

The water₁/oil emulsion mentioned above may be prepared by any suitable means for provision to the first step of the above method. For example:

(IA) an aqueous solution comprising the first drug, an osmotic agent and the hydrophilic or amphiphilic carrier matrix may be prepared;
(IB) an oil mixture comprising an organic solvent, one or more hydrophobic polymers and, optionally, a flotation agent may also be prepared; and
(IC) the aqueous solution is then added dropwise to the oil mixture to form a water in oil (i.e. water₁/oil emulsion) using standard techniques, such as agitation by any suitable method (e.g. magnetic stirring, overhead mechanical stirring, a mechanical shaker, etc.). If the flotation agent does not form part of the oil mixture in the initial preparation stage (IB), it may be added at the same time as, or after, the addition of the aqueous solution to the oil mixture. As will be appreciated, the first drug may be hydrophilic and may comprise one or more drugs (e.g. 1, 2, 3, 4 or 5 drugs). In embodiments of the invention where a second drug that is hydrophobic is desired in the resulting microcapsules, said second drug may be added to the oil mixture (IB) before the addition of the aqueous solution to said mixture. Again, the second drug may be one or more drugs (e.g. 1, 2, 3, 4 or 5 drugs). As will be noted, this method conveniently enables the first and second drugs (when hydrophilic and hydrophobic, respectively) to be contained within a specific compartment of the initial water₁/oil emulsion. As will be appreciated, the agitation may be continued for any suitable period of time to form the desired emulsion. For example, the agitation may be carried out for a time period in the range of from about 5 minutes to about 12 hours, such as from about 15 minutes to about 8 hours, from about 30 minutes to about 6 hours, from about 1 hours to about 4 hours, about 3 hours to about 6 hours, about 5 hours, about 4 hours or about 3 hours. In various embodiments, the agitation may be carried out for a time period in the range of about 3 hours to about 5 hours. The water₁/oil emulsion may be prepared just before addition to step (b) or may be prepared well in advance and, potentially, even transported from one site to a distant site for use in the disclosed method.

The water₁ phase will contain a suitable amount of drug to obtain a suitable concentration of the hydrophilic drug(s) to obtain the desired amount of the drug(s) in question in the final product. For example, each hydrophilic drug may be present in the water₁ phase in an amount of from 0.1 mg/mL to 100 mg/mL, such as from 0.5 mg/mL to 40 mg/mL, such as from 2.5 mg/mL to 25 mg/mL, such as 5 mg/mL, 10 mg/mL or 20 mg/mL.

The oil phase will contain a suitable amount of drug to obtain a suitable concentration of the hydrophobic drug(s) to obtain the desired amount of the drug(s) in question in the final product. For example, each hydrophobic drug may be present in the oil phase in an amount of from 0.1 mg/mL to 50 mg/mL, such as from 0.5 mg/mL to 40 mg/mL, such as from 2.5 mg/mL to 30 mg/mL, such as from 5 mg/mL to 25 mg/mL such as 20 mg/mL or 25 mg/mL.

Any suitable concentration of the hydrophilic or amphiphilic carrier matrix material may be used in the water₁ phase. Examples of suitable concentrations include, but are not limited to a concentration of from 1 mg/mL to 100 mg/mL, such as from 5 mg/mL to 75 mg/mL, such as from 10 to 50 mg/m L. As will be appreciated the amount of the hydrophilic or amphiphilic carrier matrix material will affect the controlled release rate profile of the resulting microcapsules and may also affect the drug loading efficiency of the hydrophilic and/or hydrophobic drugs. This is because, without a core matrix (i.e. hydrophilic or amphiphilic polymer matrix), the hydrophilic drug(s) can only be localized within the hydrophobic capsule shell in the final product (if at all). While the hydrophilic drug is entrapped in the W₁ phase during the initial phase of the preparation process, the W₁ phase is evaporated to form the final formulation. If there is no core matrix in the W₁ phase (or more properly, at the boundary of the W₁/Oil phases) to hold onto the hydrophilic drug during the evaporation of the W₁ phase, then the hydrophilic drug may leech out through the hydrophobic polymer matrix and be lost to the composition during this evaporation step. Thus, a higher concentration of the core matrix material enhances the encapsulation efficiency of hydrophilic drugs. However, while increasing the concentration of the core matrix material will increase encapsulation efficiency, this will increase the mass of the resulting composition, which may affect ability of the composition to float. Therefore, it is important to optimise the concentration of the core matrix within the composition to obtain the best possible encapsulation efficiency, while still retaining the ability to float for the required amount of time (as discussed herein). For example, as shown in the following examples section, increasing the amount of the hydrophilic or amphiphilic carrier matrix in the preparation (and hence in the core of the resulting microcapsules) may actually lead to an increase in the release rate of any hydrophobic drugs entrapped in the hydrophobic shell of the microcapsules, while not necessarily leading to a significant change to the hydrophilic molecule release rate. While not wishing to be bound by theory, this perhaps counter-intuitive effect may be because the hydrophilic/amphiphilic polymer in the core may cause a higher influx of water into the core, leading to an increase in the size and number of pores and thereby increasing the release rate of the hydrophobic drugs in the shell.

Any suitable concentration of the flotation agent may be used in the oil phase. However, it is desired that the ratio of flotation agent to organic solvent (v/v) is kept as low as possible, but yet still sufficient to provide the resulting microcapsules with adequate buoyancy. Examples of suitable concentrations for the flotation agent in the oil phase (e.g. to the organic solvent) may be from 0.01 to 2% v/v, such as from 0.05 to 1% v/v, such as from 0.1 to 0.3% v/v, such as from 0.15 to 0.2% v/v.

Any suitable volatile organic solvent (at standard ambient temperature and pressure) may be used as the organic solvent. Suitable solvents may include, but are not limited to an organic solvent selected from one or more of the group consisting of dichloromethane, chloroform, toluene, pentane, hexane, heptane, octane, nonane, n-decane, n-dodecane, benzyl chloride, hexadecane, diethyl ether, ethyl acetate, cyclohexane, chloromethane, trichloroethylene (TCE), benzene, bromodichloromethane, vinyl chloride, trichloroethane, methyl ethyl ketone, methyl isobutyl ketone, methyl tert-butyl ether, vinyl acetate, dichloroethane, chloroethane, trichlorotrifluoroethane, ethylbenzene, and isopropylbenzene, optionally wherein the organic solvent is dichloromethane.

As discussed in step (b) of the above process, the water₁/oil emulsion is then added to an aqueous solution that has a pH of from 2 to 6 under conditions suitable to form a water₁ in oil in water₂ (water₁/oil/water₂) emulsion (e.g. dropwise addition of the water₁/oil emulsion into the aqueous solution with a suitable form of agitation). As noted above, any period of time under agitation that: provides the desired water₁/oil/water₂ emulsion; and allows a portion of the organic solvent to be evaporated at ambient temperature and pressure may be used, such as at least 5 minutes under agitation (e.g. a stirrer, such as a magnetic or overhead mechanical stirrer operating at from 50 to 2,000 rpm, such as from 100 to 1,500 rpm, such as from 200 to 1,000 rpm, such as from 300 to 750 rpm, such as 400 to 600 rpm). For example, the agitation may be carried out for a time period in the range of from about 5 minutes to about 12 hours, such as from about 15 minutes to about 8 hours, from about 30 minutes to about 6 hours, from about 1 hours to about 4 hours, about 3 hours to about 6 hours, about 5 hours, about 4 hours or about 3 hours. In various embodiments, the agitation may be carried out for a time period in the range of about 3 hours to about 5 hours. As noted above, the agitation and time used may result in a portion of the organic solvent evaporating, which may in turn help to harden or precipitate the hydrophobic polymers in the oil phase.

As will be appreciated, the formation of the water₁/oil/water₂ emulsion results in water₁/oil emulsified droplets in a water₂ phase. The core of these droplets is the water₁ phase, which comprises the first drug (as defined above), the hydrophilic or amphiphilic carrier matrix material and the osmotic agent, while the oil phase comprises the hydrophobic polymers to form the shell, along with the flotation agent and, if present, the second drug (as defined above). In embodiments where the amphiphilic carrier matrix material is present, it may conveniently arrange itself at the boundary between the water₁ and oil phases. A similar effect may also occur with a hydrophilic polymer, though to a lesser extent, depending on the hydrophilicity of said hydrophilic polymer.

The stirring/agitation speed may also affect the size of the water₁ emulsion droplets in the water₁/oil emulsion and the water₁/oil droplets in the water₁/oil/water₂ emulsion. For example, the size of the emulsion droplets formed at each stage (and hence the core/shell dimensions of the resulting microparticles) may be approximately inversely proportional to the speed of stirring/agitation.

The osmotic agent included in the water₁ phase is intended to draw water from the water₂ phase through the oil phase and into the water₁ phase. As the pH of the water₂ phase is acidic (i.e. a pH value of from 2 to 6, such as from 3 to 5, such as 4), it may have the effect of precipitating or hardening the hydrophilic or amphiphilic carrier matrix material around to aid in the formation of the desired hollow core shell microcapsule. For example, the amphiphilic polymer casein may precipitate at a pH value of from 2 to 6, such as 4. The osmotic agent may be provided in the water₁ phase in a suitable amount to favour the ingress of water from the water₂ phase into the water₁ phase. Suitable concentrations of the osmotic agent may be, for example, from 0.1 mg/mL to 10 mg/mL, such as from 0.5 mg/mL to 5 mg/mL, such as from 1 to 2 mg/mL. In addition, the amount of osmotic agent used may have an effect on the size of the hollow core. As such, the size of the hollow core may be approximately directly proportional to the concentration of the osmotic agent used.

The pH of the aqueous solution that is used to form the water₂ phase may be achieved by any suitable means. For example, by the addition of mineral or organic acids to the water phase. In certain embodiments that may be mentioned herein, the acidic pH value may be obtained through including polyvinyl alcohol in the aqueous solution that forms the water₂ phase. For example, the aqueous solution of the water₂ phase comprises PVA in a concentration to provide an aqueous solution having a pH value of from 2 to 6, such as from 3 to 5, such as 4. PVA has a good hydrophilic-lipophilic balance (HLB) value of 18, is cheap and non-toxic, making it a particularly useful material to use to adjust the pH balance and/or act as a surfactant for the manufacture of a composition for consumption by a human or animal subject.

The total volume to volume ratio of the organic solvent to the aqueous solution that forms the water₂ phase may be any suitable value that will result in the formation of a water₁/oil/water₂ emulsion. For example, the total volume to volume ratio of the organic solvent to the water₂ phase may be from 3 to 50% v/v, such as from 5 to 25% v/v, such as from 12 to 20% v/v, such as 15% v/v. As will be appreciated, the volume to volume ratio selected will in part depend on the organic solvent selected, as the volume to volume ratio should be one that is more than the solubility of the organic solvent in water. In certain embodiments, the aqueous phase may further comprise an amount of the selected organic solvent that is greater than or equal to the solubility of the selected organic solvent in water, as this will reduce the solvent extraction rate during emulsification step (b) by saturating the continuous aqueous phase (i.e. the water₂ phase) in said step. In certain embodiments that may be discussed herein dichloromethane may be used as the organic solvent. As dichloromethane has a solubility of around 2% v/v, the oil/water₂ ratio may be set at 15% v/v or above. In addition, the aqueous solution that forms the water₂ phase may be pre-saturated with dichloromethane (i.e. comprise around 2% v/v/dichloromethane) before the water₁/oil emulsion is added in step (b) above. For the avoidance of doubt, the volume of the water₂ phase is based on the combined volume of water and any other materials dissolved therein (e.g. PVA and any organic solvent that has been added to pre-saturate the aqueous solution).

Steps (c) and (d) of the above process are used to accelerate the precipitation rate of the capsule polymers (both hydrophilic and hydrophobic). This is achieved through both the addition of a second aqueous solution in step (c) and by the use of reduced pressure under a centrifugal force to remove yet more of the organic solvent in step (d). In step (c) of the above process, any suitable additional volume of water may be added. For example, the volume to volume ratio of the second aqueous solution to first aqueous solution may be from 1:1 to 10:1, such as from 2:1 to 5:1, such as 3:1. As will be appreciated, the second aqueous solution added in step (c) may conveniently be essentially identical to the first solution. For example, the second aqueous solution may comprise PVA in a concentration to provide an aqueous solution having a pH value of from 2 to 6, such as from 3 to 5, such as 4.

One notable feature of the currently disclosed process is that it makes it easy to encapsulate more than one drug into the resulting microcapsules, even when the drugs have very different polar properties (i.e. one or more drugs are hydrophilic, while the one or more other drugs are hydrophobic). With that in mind, it is possible to distribute both hydrophobic and hydrophilic drugs within specific compartments of the microcapsules disclosed herein in a simple formulation process. In other words, following the above method, the first (hydrophilic) drug is distributed in the carrier matrix within the hollow core, while the second (hydrophobic) drug is distributed in the hydrophobic shell of the microcapsules. Thus, in embodiments that may be mentioned herein, the process allows:

(ab) the hydrophilic drugs levodopa (LD) and carbidopa (CD) to be distributed in the carrier matrix and the hydrophobic drug entacapone (ENT) to be distributed in the shell;
(bb) the hydrophilic drug metformin (MET) to be distributed in the carrier matrix and the hydrophobic drug fenofibrate (FEN) to be distributed in the shell; or
(cb) the hydrophilic drugs comprises isoniazid (ISO) and ethambutol (ETH) to be distributed in the carrier matrix and the hydrophobic drug rifampicin (RIF) to be distributed in the shell.

In particular embodiments of the invention, the process may involve: dissolving casein in distilled water with sodium chloride as an osmolyte (osmotic agent); dissolving PLLA and

PCL in dichloromethane to obtain a PLLA/PCL polymer solution; dissolving hydrophilic drug/s (e.g. the hydrophilic drugs mentioned above) in the casein solution, while adding hydrophobic drug/s (e.g. the hydrophobic drugs mentioned above) into the PLLA/PCL polymer solution; introducing the resultant casein solution drop-wise into the resultant polymer solution with further addition of fish oil under stirring to form the primary water-in-oil (W/O) emulsion; and dispersing the W/O emulsion into a polyvinyl alcohol solution and stirring to obtain the microcapsules (e.g. with the application of reduced pressure and centrifugal force).

As will be appreciated, the resulting hollow core-shell microcapsules have a casein-loaded core, where the casein is coated on the walls of the cavity in the hollow core-shell microcapsule.

Focusing on specific process variables discussed above allows the skilled person to achieve:
1. the specific localization of hydrophobic and hydrophilic drugs compartmentalized in different parts of the microcapsule, i.e. carrier matrix-loaded (e.g. casein-coated) walls of the hollow cavity and/or the hydrophobic polymer shell; and/or
2. controlled drug release kinetics by altering the amount of the carrier matrix (e.g. casein) used.

Thus, the process described herein provides a versatile and robust approach to prepare a formulation that can deliver multiple drugs, while providing controlled release in the gastric region for a prolonged duration. In other words, the fabrication method disclosed herein provides a floating microcapsule that can simultaneously encapsulate and release more than one drug (e.g. all three PD drugs) in a controlled manner. The method also improves the encapsulation efficiency of the hydrophilic drugs (e.g. increasing the loading of the carrier matrix material may increase the encapsulation efficiency of the hydrophilic drugs). As will be appreciated, the addition of too much of the hydrophilic/amphiphilic polymer may result in a more dense composition that has reduced buoyancy. Given this, it is generally desirable to provide an amount of the hydrophilic/amphiphilic polymer that will provide increased encapsulation efficiency, while retaining sufficient buoyancy. For example, when using casein as the hydrophilic/amphiphilic polymer, the amount of casein used may be sufficient to ensure that the microcapsules contain an average amount of from 0.1 to 4.9% w/w of casein. In addition, the method also offers great versatility in being able to control drug release rates by manipulating different particle parameters, i.e. capsule/coating layer thickness and polymer ratio. Unlike other methods of producing floatable delivery systems, high temperature and compression forces are not required in this technique. Instead, only simple and economical apparatus such as an overhead stirrer, a rotary evaporator etc., are required.

The invention will now be further described with reference to the following non-limiting examples.

### Materials and Methods

Casein sodium salt from bovine milk, Poly-L-lactide (PLLA) (IV: 2.4, Purac), Polycaprolactone (PCL) (molecular weight 10 kDa, Sigma-Aldrich), and Polyvinyl alcohol (PVA) (molecular weight 30-70 kDa, Sigma-Aldrich) were used without further purification. LD, CD, ENT, FEN, MET, ISO, ETH, RIF, Tween 20, HCl solution (37% v/v Fuming) and acetic acid were purchased from Sigma-Aldrich (Steinheim, Switzerland). Dichloromethane (DCM) and acetonitrile (ACN), were purchased from Tedia Co. Inc. Olive oil (Pietro Coricelli) was used. All other chemicals and reagents used were of analytical grade. The simulated gastric fluid (SGF) (pH 1) was prepared by adding 0.1 M HCl solution to 0.02% (w/v) Tween 20. The simulated intestinal fluid (SIF) was prepared by mixing pH 6.8 phosphate buffer and 0.02% (w/v) Tween 80.

### EXAMPLES

### Example 1: Preparation Procedure of Microcapsules

Encapsulation of drugs in casein-PLLA/PCL microcapsules was performed using the water-oil-water (W₁/O/W₂) double emulsion method. The amphiphilic agent, casein, is capable of encapsulating both hydrophobic and hydrophilic drugs. Casein is a major protein in milk and has distinct hydrophobic and hydrophilic domains (i.e. amphiphilic).

An aqueous casein solution was prepared by dissolving 10, 30 or 50 mg of casein in distilled water with 2 mg of sodium chloride (1 mL) as an osmolyte. Separately, a polymer solution was prepared by dissolving 0.3 g of PLLA and 0.1 g PCL in 5 mL of DCM. Depending on the target disease (see table below), specific hydrophilic drugs are dissolved in the casein solution, while hydrophobic drugs are dissolved in the PLLA/PCL solution.

| Target disease | Hydrophilic drug | Hydrophobic drug |
|---|---|---|
| Parkinson's | Levodopa (LD-20 mg) and | Entacapone (ENT-25 mg) |
| disease | carbidopa (CD-5 mg) | |
| Diabetes | Metformin (MET-20 mg) | Fenofibrate (FEN-20 mg) |
| Tuberculosis | Isoniazid (ISO-10 mg) and | Rifampicin (RIF-20 mg) |
| | ethambutol (ETH-10 mg) | |

The resultant casein solution was then introduced drop-wise into the polymer solution with a further addition of 10 μL of fish oil under magnetic stirring. The drug-loaded, casein-containing solution was emulsified in the PLLA/PCL solution under magnetic stirring to form a primary W/O emulsion. This emulsion was then further dispersed into a solution containing 0.25% (w/v) aqueous PVA (pH 4.0, 50 mL) and DCM (1 mL) to form a W₁/O/W₂ emulsion, with an over-head stirrer (Calframo BDC1850-220). The stirrer was operated at 400 rpm for 10 mins to accelerate the evaporation of DCM, and to harden the casein at pH 4.0.

The resultant emulsion was quickly added to a round bottom flask filled with 0.25% (w/v) aqueous PVA solution (150 mL) and transferred to a rotary evaporator to solidify the microcapsules through the quick evaporation of DCM for 0.5 h. The microcapsules obtained were then centrifuged, washed with distilled water for three times and freeze dried for further use.

The release rates of the hydrophilic drugs can be adjusted by varying the casein concentration in the microcapsules. The casein concentrations are 10, 30 or 50 mg (or 1%, 3% or 5% w/v relative to the initial aqueous solution).

### Effect of Process Parameters on Microcapsules Properties

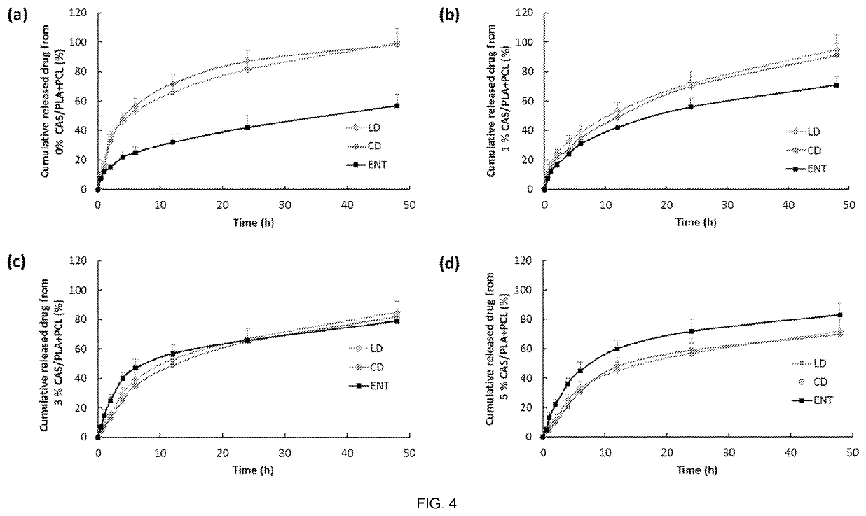

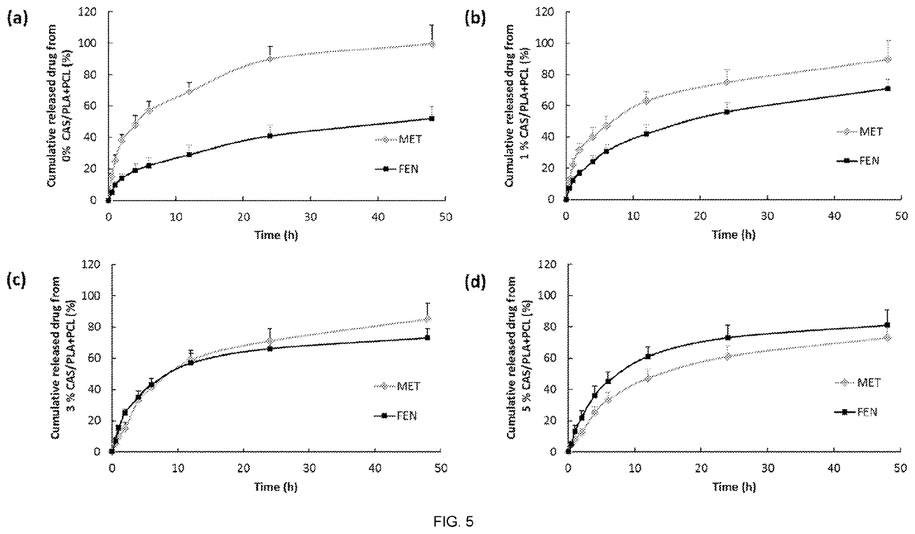

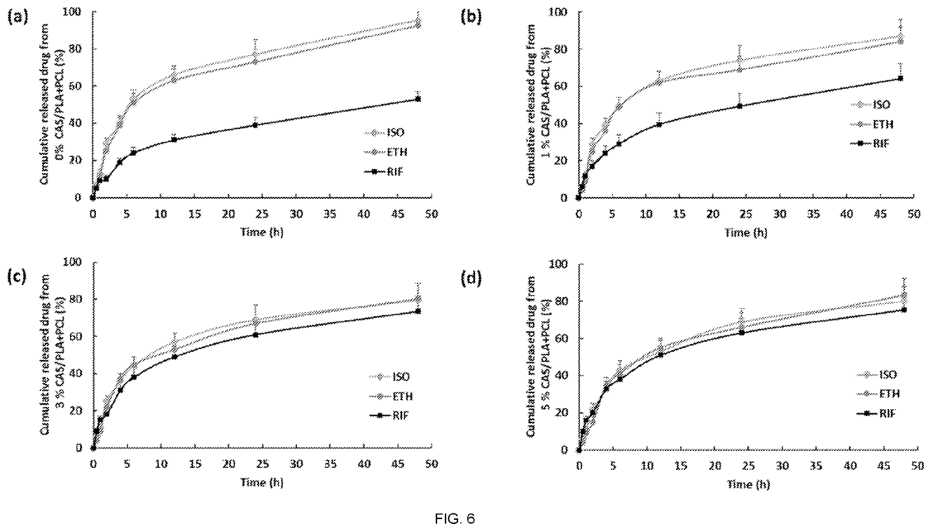

1. Concentration of casein: It can be manipulated to control release rates and profile. Increasing casein concentration would increase the drug loading efficiency of hydrophilic drugs (see Table 1) and increase the drug release rates of hydrophobic drugs (see FIGS. 4 to 6). Similar effects may be achieved using different hydrophilic and, particularly, amphiphilic polymeric materials.
2. O/W₂ volume ratio: It should be kept far above the solubility of organic solvent in water. For example, solubility of dichloromethane (DCM) in water is 2% v/v, so the O/W₂ ratio should be set at 10% v/v or above.
3. Composition and ratio of polymers in capsule shell: This is to modify capsule morphology and level of porosity to influence drug release rates. E.g. ratio of PLLA and PCL was kept at 3:1.

### SEM Images of Drug-Loaded Microcapsules

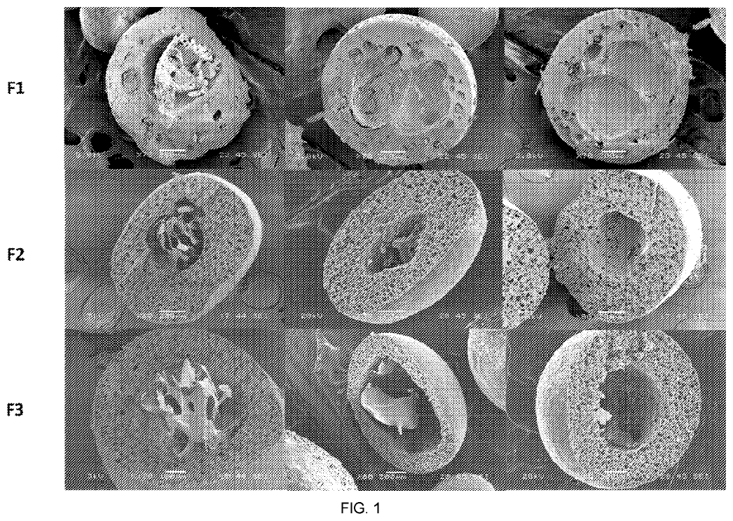

FIG. 1 shows the scanning electron microscopy (SEM) images of the microcapsules fabricated. Regardless of the polymer blend ratios, all microcapsules prepared were spherical in shape and around 600 μm in size.

FIG. 1 also shows the hollow structure of the microcapsules, which helps to achieve better floatability (that is, lower density), thus providing prolonged gastric residence time of these microcapsules. The microcapsules with hollow cavities were obtained with the use of a rotary evaporator under reduced pressure, which provides a fast solvent extraction rate.

### Encapsulation Efficiency

To determine the best encapsulation efficiency and optimized release profile of the drugs, three different concentrations of casein (1, 3 and 5% w/v) were loaded in the microcapsules. When used herein, reference to w/v percentage of casein is intended to refer to the w/v percentage in the water₁ phase used to manufacture the resulting compositions. As such, 1% w/v casein above means that the water₁ phase used to manufacture the resulting composition contained 1% w/v of casein. This was repeated for three sets of drugs meant for the three targeted diseases, thus altogether a total of nine microcapsule samples were prepared. These samples are entitled F1 to F9. Table 1 shows the encapsulation efficiency of various drugs as a function of casein loading in the samples (as will be appreciated the w/v % listed refers to the w/v % of casein in the water₁ phase used to manufacture the formulation).

The procedure to measure the amount of drug in each microcapsule sample is provided as follows. Microcapsules (10 mg) were accurately weighed and dissolved in 1 ml of DCM through sonication. SGF (10 mL) was then added, and mixed using a vortex at 300 rpm (n=3). The hydrophilic LD and CD partitions into SGF and the supernatant was analyzed using Reverse Phase High Performance Liquid Chromatography (RP-HPLC) with 100% Acetic Acid (2% v/v) as mobile phase at wavelength 284 nm. For measuring MET, the mobile phase was acetonitrile:potassium phosphate buffer in water (40:60 v/v) at wavelength 252 nm. ISO and ETH were co-analyzed with 20 mM monobasic sodium phosphate buffer and acetonitrile at 210 nm. The ENT is then re-dissolved in Sodium Dihydrogen Phosphate (60%)/Methanol (40%) Mixture to precipitate polymer. The supernatant is then taken and filtered through a 0.22 μm syringe filter. The resultant solution is then analyzed using RP-HPLC with Sodium Dihydrogen Phosphate (60%)/Methanol (40%) Mixture as mobile phase at wavelength 284 nm. FEN was analyzed with acetonitrile (70%)/Water (30%) at 295 nm. RIF was detected with acetonitrile (60%)/monopotassium phosphate (0.075 M, 40%) at wavelength of 254 nm. The following equation was applied to calculate the encapsulation efficiency:

Encapsulation efficiency (%)=Measured amount of drug in the microcapsules/Weight of the used drug×100%

TABLE 1
Encapsulation efficiency (%) of various drugs
in different casein/PLLA + PCL microcapsules.

| | Control | | | |
|---|---|---|---|---|
| | 0% (w/v) | 1% (w/v) | 3% (w/v) | 5% (w/v) |
| | casein/ | casein/ | casein/ | casein/ |
| | PLLA + | PLLA + | PLLA + | PLLA + |
| | PCL | PCL | PCL | PCL |
| PD drugs | Control I | F1 | F2 | F3 |
| LD | 12.5 ± 4.7 | 31.3 ± 6.6 | 43.5 ± 4.1 | 55.7 ± 7.5 |
| CD | 15.5 ± 5.1 | 29.3 ± 5.3 | 41.0 ± 6.5 | 59.1 ± 4.3 |
| ENT | 79.5 ± 6.3 | 77.1 ± 4.8 | 75.2 ± 3.3 | 73.7 ± 7.2 |
| Diabetes drugs | Control II | F4 | F5 | F6 |
| FEN | 89.3 ± 4.7 | 84.1 ± 5.2 | 82.7 ± 6.3 | 80.3 ± 4.9 |
| MET | 10.7 ± 5.5 | 21.4 ± 3.9 | 34.2 ± 6.7 | 48.7 ± 4.1 |
| TB drugs | Control III | F7 | F8 | F9 |
| ISO | 19.3 ± 6.1 | 28.5 ± 4.3 | 39.1 ± 4.7 | 60.3 ± 4.2 |
| ETH | 21.5 ± 5.7 | 31.2 ± 3.2 | 44.3 ± 4.7 | 58.9 ± 7.7 |
| RIF | 82.5 ± 8.3 | 80.1 ± 6.6 | 81.5 ± 3.5 | 80.3 ± 6.2 |

It is clear from Table 1 that drug loading efficiency of hydrophilic drugs increases with increasing casein concentration. The hydrophilic drugs are levodopa (LD), carbidopa (CD), metformin (MET), isoniazid (ISO, Log P: −0.7) and ethambutol (ETH, Log P: −0.14). Rifampicin is a hydrophobic drug, having a Log P of 4.24.

### Buoyancy

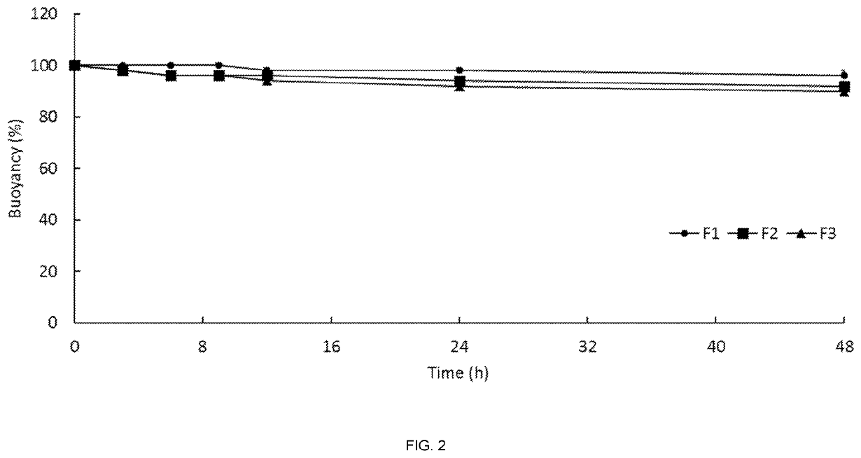

The buoyancy of the microcapsules was tested by visual inspection. The samples were considered buoyant only if more than 90% of microcapsules remained afloat after the prescribed test time (48 h) in simulated gastric fluid (SGF) (pH 1) at 37° C. under constant agitation of 250 rpm using a magnetic stirrer (FIG. 2).

It was found that more than 90% of the microcapsules remained afloat even up to 48 hours (when the test was stopped). Specifically, the buoyancy (%) of F1, F2 and F3 was found to be 98, 92 and 90%, respectively.

### Raman Mapping

To determine the polymer localization within the microcapsules, Raman mapping was conducted for F2, a 3% (w/v) casein-loaded microcapsule, as a representative sample. Raman mapping was used for observing the polymer and drugs distribution within the microcapsules. Cross-sectioned microcapsule was put under a microscope objective with a laser power of 10 mW. Raman mapping measurements were carried out with a step size interval of 5 μm to form a grid map using a Raman microscope (Nicolet™ isTM50, Thermo Scientific) equipped with a near-infrared enhanced deep depleted thermoelectrically Peltier-cooled CCD array detector and a high-grade Leica microscope. The pre-sectioned microcapsule was irradiated with a 785 nm near-infrared diode laser, and the back scattered light was collected by an objective lens. Measurement scans were collected in a spectrum range from 200 to 3200 cm⁻¹.

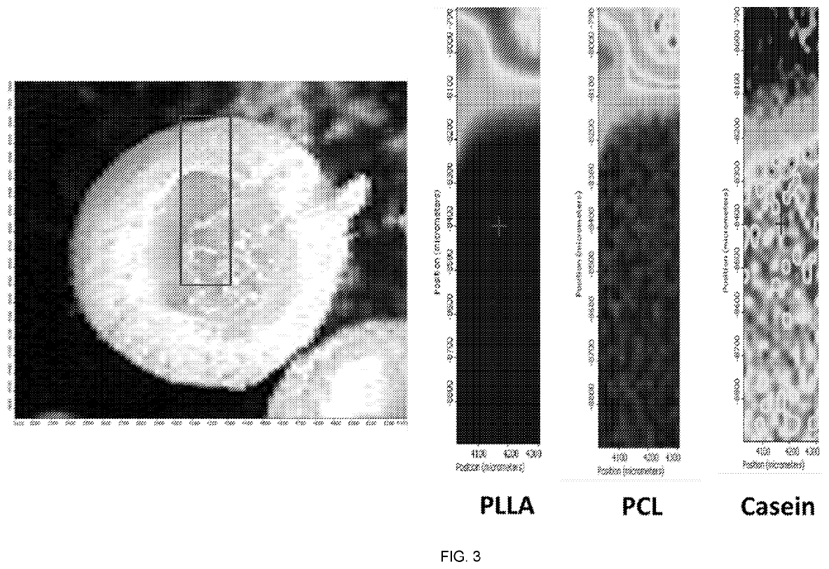

Raman mapping shows that, while a high intensity of PLLA and PCL were observed in the shell of the microcapsule, the casein was uniformly distributed in the hollow cavity at the core of the microcapsule (see FIG. 3).

### Example 2: In-Vitro Drug Release Profile of Microcapsules

To test the hypothesis that the microcapsules can provide better sustained and controlled release of multiple drugs, the release profiles of the individual drugs from the samples in simulated gastric fluid (SGF) and simulated intestinal fluid (SIF) were investigated.

### Method

In vitro release study was conducted in SGF for 48 h. In some experiments, the study was conducted in SGF, followed by SIF, where the total duration in SGF and SIF is 48 h. Each microcapsule sample (20 mg) was added to a bottle containing SGF or SIF medium (20 mL). Both SGF and SIF bottles were placed in a 37° C. rotating incubator. At different time points, half of the medium (10 mL) in each bottle was extracted and replaced with 10 mL new medium. The time points included intervals of hours within a 48-hour period. The drug content in the extracted medium was analyzed using RP-HPLC with the procedure mentioned in Example 1 for determining encapsulation efficiency.

### Results

As observed from FIGS. 4 to 6, the release rates of the hydrophilic drugs (i.e. LD, CD, MET, ISO, ETH) in SGF were independent of casein concentration. On the other hand, the release rate of hydrophobic drugs (i.e. ENT, FEN, RIF) encapsulated in the capsule shell increased with higher concentration of casein.

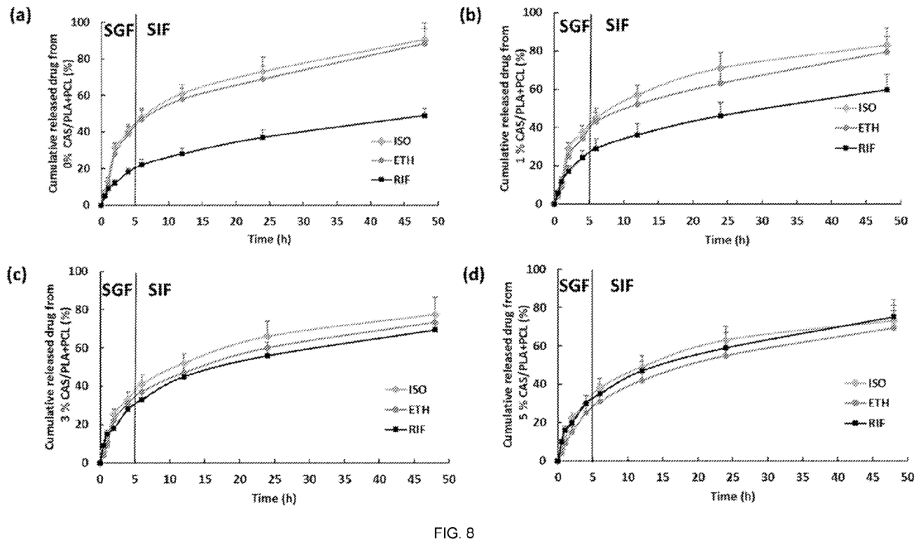

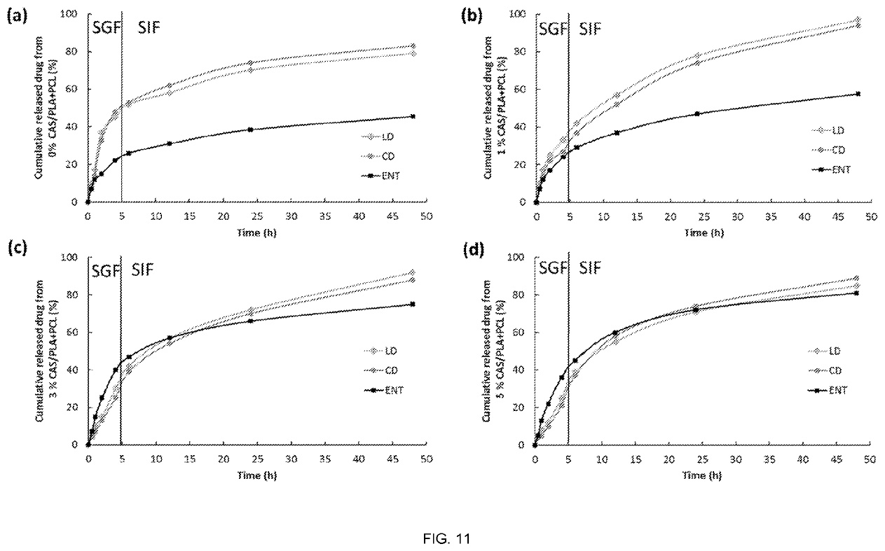

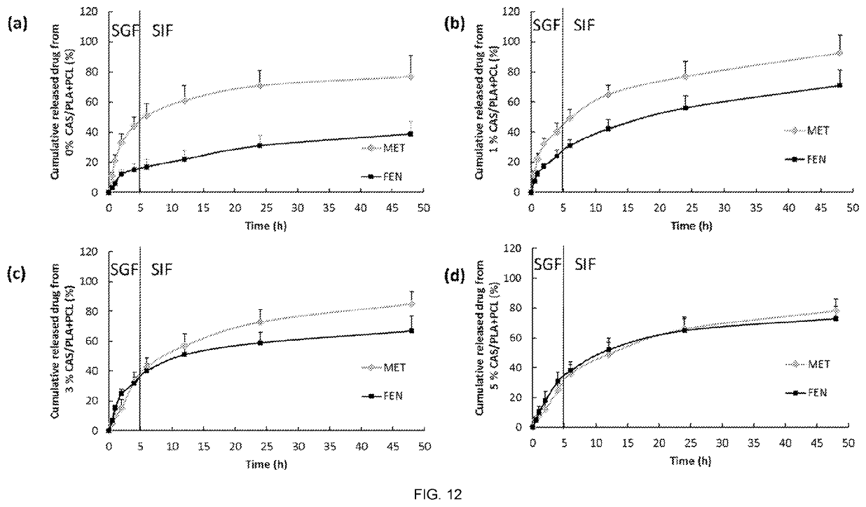

As floating microspheres are able to provide prolonged GRT of 5 h in the human body, a further release study was conducted but, this time, in an environment that would simulate drug release in the stomach, based on typical gastric emptying time, followed by release in the intestinal region. The intent of this study was to show that (however unlikely) even if the microcapsules enter the intentines, they are still capable of providing sustained release. As such, microcapsule samples were added to SGF for 5 h, followed by SIF for the remaining duration up to 48 h. As observed from FIGS. 8, 11 and 12, the samples exhibited relatively similar release rates as compared to samples kept entirely in SGF. Since the gastrointestinal absorption site of all drugs used in this work was reported to be the stomach and upper intestine, having a sustained release of drugs in the stomach would be advantageous.

### Water Uptake

The effect of increasing casein content on the water uptake of microcapsules was also investigated.

Microcapsules were weighed (50 mg) and placed in glass bottles filled with SGF (20 mL). Samples were incubated at 37° C. with gentle shaking. At pre-determined time points, microcapsules were collected from the bottles. For water uptake study, the microcapsules were washed with distilled water, weighed, and dried to obtain the dry mass. The percentage of water uptake was calculated at pre-determined time point as the difference between the mass of the wet and dry microcapsules, measured at time t, and taken as a percentage of the dry weight. Each experiment was conducted in triplicate. Molecular weight of the microcapsules was measured using the Agilent GPC 1100 Series using a reflective index detector (RID) at 30° C. Chloroform used as solvent and the flow rate was 1 mL/min. Based on the solubility differences of the polymers in THF (PCL is soluble in THF, while PLLA is not), the two polymers in the microcapsules were separated by the dissolution method. Each microcapsules (10 mg) were added in THF (1 mL) to dissolve the PCL. The mixture solution was evaporated at room temperature for 48 h. The remaining solvent in the solution was further dried in an oven at 40° C. for a 48 h. And then, chloroform (1 mL) added to dried PLLA and analyzed for GPC. Molecular weights of the microcapsules were calculated by the calibration curve using polystyrene standards (165-5000 kDa).

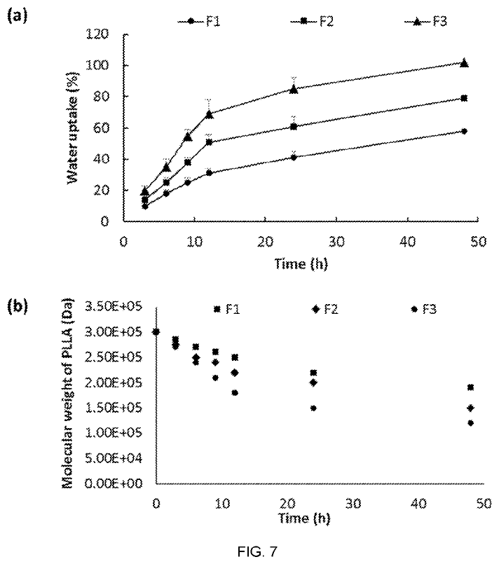

Increasing casein content increases water uptake into the microcapsule, resulting in an accelerated release of hydrophobic drugs—the increase in water uptake over time is shown in FIG. 7a.

The increase in water uptake also accelerates PLLA and PCL polymer degradation (see FIG. 7b).

### Example 3: In-Vivo Drug Release Profile of Microcapsules

Pharmacokinetics of three different PD drugs (i.e. levodopa, carbidopa and entacapone) released from optimized casein (3% (w/v))-microparticles that were fed to healthy mice, was compared against a commercial formulation having an identical drug ratio to those in the microcapsules (i.e. control).

### Methods

The experimental mice were divided into two groups (control and F2) each comprising of five animals. Control formulation (as a solution) was prepared fresh each experimental day in 0.6% methyl cellulose diluted with saline solution. Mice were subsequently administered with 200 ul (control) or 300 μl (pellet) single dose of drug solution at LD:CD:ENT=10:2.5:20 mg·kg⁻¹ (Group 1, conventional formulation) and LD:CD:ENT=10:2.5:20 mg·kg⁻¹ (Group 2, F2 via oral gavage using a feeding syringe. At stipulated time points of 0.25, 0.5, 1, 2, 4, 8, 12, 24 hr, the mice were euthanized and blood was collected via cardiac puncture with ethylenediaminetetraacetic acid (EDTA) as the anticoagulant. The blood samples were then centrifuged (4,500 rpm) for 10 min at 25° C. to obtain the plasma. In addition, the brain was harvested and flash freeze in liquid nitrogen. Samples were stored in −80° C. before further analysis via LC/MS. Analysis of drugs in plasma and brain was conducted using LC/MS (Ribeiro et al., (2015). Bioanalysis, 7, 207-220). An Agilent 1290 HPLC system with an Agilent 6120 Quadrupole Mass Spectrometer was used to measure plasma and brain concentrations of drugs. The mobile phase consisted of a gradient of (A) 0.1% (v/v) formic acid (FA) and a mixture of ACN:MeOH (90:10, v/v) containing 0.1% (v/v) FA.

TABLE 2
The gradient profile of (A) 0.1% (v/v) formic
acid and (B) ACN:MeOH (9:1, v/v).

| Time (min) | A (%) | B (%) | Flow rate (mL/min) |
|---|---|---|---|
| 0 | 100 | 0 | 1 |
| 2 | 98 | 2 | 1 |
| 2.1 | 10 | 90 | 1 |
| 3.5 | 10 | 90 | 1 |
| 3.6 | 98 | 2 | 1 |
| 8.0 | 98 | 2 | 1 |

The gradient elution is tabulated in Table 2. XBridge C8 column (150×4.6 mm; particle size 5 μm) was used at 30° C. The injection volume of samples was 20 μl. Plasma and brain extracted solution were mixed with internal standard and extracted by solid-phase extraction. The calibration curves were linear over the range of 2 to 2000 ng/mL for LD, 2 to 400 ng/mL for CD and 5 to 3000 ng/mL for ENT.

### Drug Plasma Concentration

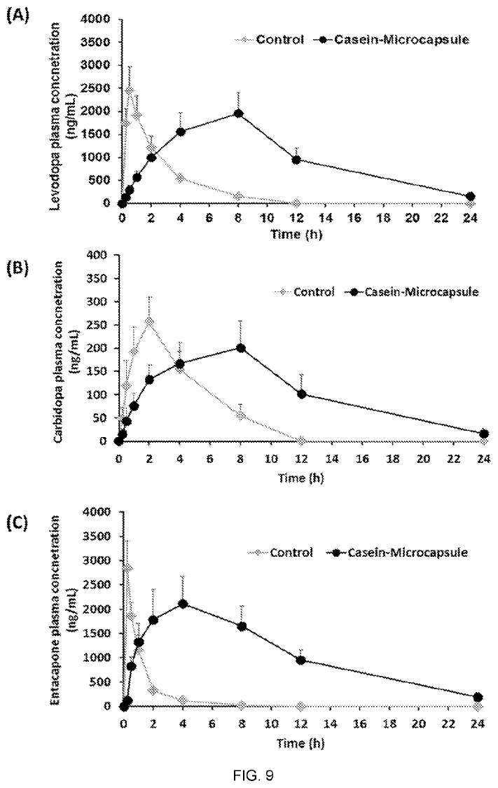

FIG. 9 shows that casein-microparticles showed sustained release of all three PD drugs (i.e. levodopa, carbidopa and entacapone) when compared to the control, based on the respective drug concentrations in blood plasma. These results confirm the sustained releasing capability of a delivery system based on the microcapsules, and in prolonging the action of the drugs in vivo.

The pharmacokinetic data for each drug is reflected in Table 3 below. The data shows that the mean residence time (MRT) of levodopa increased from 2.6 hrs to 10.1 hrs=˜4× increase. The bioavailability (AUC) of levodopa was observed to increase by 2.8×.

TABLE 3
Pharmacokinetic parameters of levodopa, carbidopa and entacapone from control and casein-microcapsule in the plasma (n = 5).

| | Levodopa | | Carbidopa | | Entacapone | |
|---|---|---|---|---|---|---|
| | Control | Casein-microcapsule | Control | Casein-microcapsule | Control | Casein-microcapsule |
| cₘₐₓ (ng/ml) | 2479.9 ± 454.2 | 1955.1 ± 398.5 | 342.3 ± 98.5 | 201.0 ± 57.1 | 3289.2 ± 571.6 | 2111.5 ± 554.6 |
| tₘₐₓ (h) | 0.5 | 8 | 2 | 8 | 0.3 | 4 |
| t½(h) | 1.9 ± 0.4 | 10.3 ± 2.1 | 5.4 ± 1.1 | 12.6 ± 2.9 | 0.5 ± 0.1 | 10.5 ± 2.1 |
| AUC_{0-∞} | 7652.2 ± 1052.7 | 21580.4 ± 3957.4 | 1402.7 ± 281.4 | 3542.1 ± 811.7 | 6514.8 ± 621.2 | 18554.6 ± 6842.9 |
| (h · g/ml) | | | | | | |
| CL (ml/h/kg) | 1341.4 ± 319.6 | 576.7 ± 196.3 | 1798.2 ± 311.9 | 721.4 ± 139.6 | 8324.5 ± 2247.8 | 721.5 ± 235.4 |
| MRT (h) | 2.57 ± 0.89 | 10.1 ± 2.11 | 3.35 ± 1.15 | 9.41 ± 2.24 | 0.74 ± 0.15 | 7.12 ± 2.82 |

### Brain Concentrations of Levodopa and Dopamine

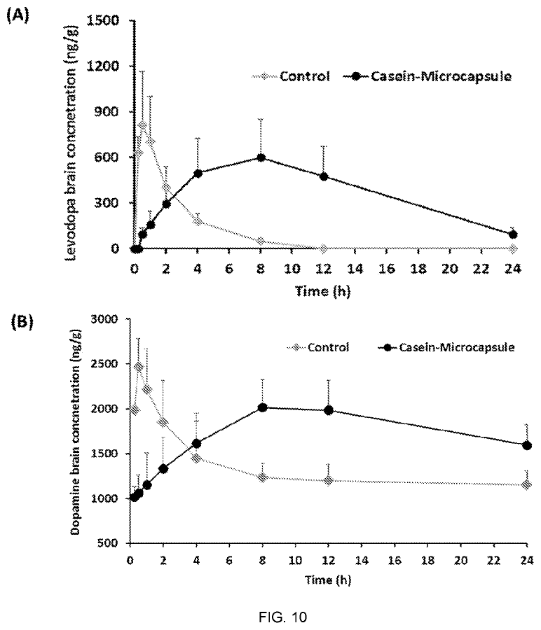

FIG. 10 shows that a prolonged elevation of dopamine was observed for mice fed with the drug-loaded casein-microparticles as compared to the control. This confirms the conversion of levodopa into dopamine in the brain of the mice as a consequence of higher bioavailability of levodopa This validates the effectiveness of this delivery system in:
1. Controlling and sustaining the release of multiple PD drugs.
2. Allowing for increased MRT and bioavailability of PD drugs.
3. Converting levodopa into dopamine as shown from the prolonged elevated levels of dopamine in the brain of mice.

1. A sustained release hollow core-shell microcapsule formulation for drug delivery, comprising:
a hollow shell having an outer surface and an inner surface that is formed from one or more hydrophobic polymers;
a hydrophilic or amphiphilic carrier matrix distributed over the inner surface of the hollow shell;
a first drug distributed within the hydrophilic or amphiphilic carrier matrix;
optionally, an osmotic agent; and
a flotation agent, wherein
the microcapsule is capable of floating in a simulated digestive fluid for a period of from 24 to 96 hours.

2. The microcapsule according to claim 1, wherein the hydrophobic polymer is selected from one or more of the group consisting of poly(L,D-lactic-co-glycolic acid) (PLGA), poly(L-lactide) (PLLA), poly(ε-caprolactone) (PCL), poly(glycolide) (PGA), poly(lactide) (PLA), and co-polymers thereof.

3. The microcapsule according to claim 2, wherein the hydrophobic polymer is a blend of PLLA and PCL.

4. (canceled)

5. The microcapsule according to claim 1, wherein the ratio of the hydrophilic or amphiphilic carrier matrix to the hydrophobic polymer is from 1:100 to 1:3 w/w, such as from 1:50 to 1:8 w/w, such as from 1:40 to 1:10 w/w.

6. The microcapsule according to claim 1, wherein the hydrophilic or amphiphilic carrier matrix is selected from one or more of the group consisting of alginate, chitosan, casein, starch, hyaluronic acid, gelatin, agarose, collagen, fibrin, dextran, polyvinylalcohol (PVA) and polyethylene glycol (PEG).

7. The microcapsule according to claim 6, wherein the hydrophilic or amphiphilic carrier matrix is casein.

8. (canceled)

9. The microcapsule according to claim 1, wherein the microcapsule incorporates the features of one or both of:
(a) the flotation agent is an oil, wherein the oil is selected from one or more of the group consisting of fish oil, olive oil, corn oil, sunflower seed oil, grape seed oil, canola oil, avocado oil, and coconut oil; and
(b) the flotation agent is present in an average amount of from 0.1 to 10 wt % of the total weight of the microcapsule.

10-13. (canceled)

14. The microcapsule according to claim 1, wherein the first drug is a hydrophilic drug and/or the osmotic agent is an alkaline metal salt or an alkaline earth salt, such as sodium chloride.

15. The microcapsule according to claim 1, wherein the microcapsule further comprises a second drug that is hydrophobic and distributed within the hydrophobic polymer shell.

16-18. (canceled)

19. The microcapsule according to claim 15, wherein:
(aa) the first drug comprises levodopa (LD) and carbidopa (CD) and the second drug comprises entacapone (ENT);
(ba) the first drug comprises metformin (MET) and the second drug comprises fenofibrate (FEN); or
(ca) the first drug comprises isoniazid (ISO) and ethambutol (ETH) and the second drug comprises rifampicin (RIF).

20. A method of forming a sustained release hollow core-shell microcapsule formulation for drug delivery as defined in claim 1, comprising the steps of:
(a) providing a water₁/oil emulsion, where
the water₁ phase comprises a hydrophilic or amphiphilic carrier matrix material, a first drug and an osmotic agent,
the oil phase comprises an organic solvent, one or more hydrophobic polymers and a flotation agent;
(b) adding the water₁/oil emulsion to an aqueous solution having a first volume and a pH value of from 2 to 6 and agitating at ambient temperature for a period of time to form a water₁/oil/water₂ emulsion;
(c), adding a second volume of an aqueous solution having a pH value of from 2 to 6 to the water₁/oil/water₂ emulsion to form a final intermediate mixture; and
(d) subjecting the final intermediate mixture to a centrifugal force and removing the organic solvent and, optionally, the water under reduced pressure to form the hollow core-shell microcapsules.

21. The method according to claim 20, wherein the method makes use of one or more of the following features:
(i) the concentration of the hydrophilic or amphiphilic carrier matrix material in the water₁ phase is from 1 mg/mL to 100 mg/mL, such as from 5 mg/mL to 75 mg/mL, such as from 10 to 50 mg/mL;
(ii) the concentration of the osmotic agent in the water₁ phase is from 0.1 mg/mL to 10 mg/mL, such as from 0.5 mg/mL to 5 mg/mL, such as from 1 mg/mL to 2 mg/mL;
(iii) the concentration of the flotation agent in the oil phase is from 0.01 to 2% v/v, such as from 0.05 to 1% v/v, such as from 0.1 to 0.3% v/v, such as from 0.15 to 0.2% v/v;
(iv) the agitation in step (b) is provided by a stirrer operating at from 50 to 2,000 rpm, such as from 100 to 1,500 rpm, such as from 200 to 1,000 rpm, such as from 300 to 750 rpm, such as from 400 to 600 rpm;
(v) the pH of the water₂ phase is from 2 to 6, such as from 3 to 5, such as 4;
(vi) the aqueous solution of the water₂ phase comprises PVA in a concentration to provide an aqueous solution having a pH value of from 2 to 6, such as from 3 to 5, such as 4;
(vii) the water₂ phase further comprises an amount of the organic solvent greater than or equal to the solubility of said organic solvent in water;
(viii) the total volume to volume ratio of the organic solvent to the water₂ phase is from 3 to 50% v/v, such as from 5 to 25% v/v, such as from 12 to 20% v/v, such as 15% v/v; and
(ix) the organic solvent is selected from one or more of the group consisting of dichloromethane, chloroform, toluene, pentane, hexane, heptane, octane, nonane, n-decane, n-dodecane, benzyl chloride, hexadecane, diethyl ether, ethyl acetate, cyclohexane, chloromethane, trichloroethylene (TCE), benzene, bromodichloromethane, vinyl chloride, trichloroethane, methyl ethyl ketone, methyl isobutyl ketone, methyl tert-butyl ether, vinyl acetate, dichloroethane, chloroethane, trichlorotrifluoroethane, ethylbenzene and isopropylbenzene.

22. (canceled)

23. The method according to claim 20, wherein the hydrophobic polymer is selected from one or more of the group consisting of poly(L,D-lactic-co-glycolic acid) (PLGA), poly(L-lactide) (PLLA), poly(ε-caprolactone) (PCL), poly(glycolide) (PGA), poly(lactide) (PLA), and co-polymers thereof.

24-25. (canceled)

26. The method according to claim 20, wherein the hydrophilic or amphiphilic carrier matrix material is selected from one or more of the group consisting of alginate, chitosan, casein, starch, hyaluronic acid, gelatin, agarose, collagen, fibrin, dextran, polyvinylalcohol (PVA) and polyethylene glycol (PEG).

27. The method according to claim 26, wherein the hydrophilic or amphiphilic carrier matrix material is casein.

28-29. (canceled)

30. The method according to claim 20, wherein the first drug is a hydrophilic drug.

31. The method according to claim 20, wherein the oil phase of the water₁/oil emulsion further comprises a second drug that is hydrophobic.

32-33. (canceled)

34. A method of treating a chronic disease, comprising the step of administering a suitable amount of a sustained release hollow core-shell microcapsule formulation as described in claim 1 to a subject in need thereof.

35-36. (canceled)

37. The method according to claim 34, wherein the chronic disease is selected from one or more of the group consisting of Parkinson's disease, diabetes, tuberculosis, stroke, HIV, mental disorders, cancer, Alzheimer's disease, disorders of lipid metabolism, lupus, metabolic syndrome, hypertension, chronic renal failure, inflammation, lupus, obesity, atherosclerosis, angina pectoris, myocardial infarction, gastric ulcer, alcoholic liver disease, and degenerative arthritis.

38. The microcapsule according to claim 3, wherein the PLLA and PCL form a blend having a w/w/ ratio of from 5:1 to 1:5, such as 3:1.
